(19) United States
(12) Patent Application Publication
(10) Pub. No.: US 2003/0195213 A1
(43) Pub. Date: Oct. 16, 2003

(54) Compounds, compositions and methods for treating or preventing viral infections and associated diseases

(76) Inventors: Thomas R. Bailey, Upper Providence Township, PA (US); Dorothy C. Young, Upper Providence Township, PA (US)

Correspondence Address: DANN, DORFMAN, HERRELL & SKILLMAN, 1601 MARKET STREET, SUITE 2400, PHILADELPHIA, PA, 19103-2307, US

(21) Appl. No.: 10/366,796

(22) Filed: Feb. 14, 2003

Related U.S. Application Data
(63) Continuation of application No. 09/763,261, filed on Apr. 23, 2001, now abandoned.
Application No. 09/763,261 is a 371 of international application No. PCT/US99/18785, filed on Aug. 19, 1999.
(60) Provisional application No. 60/097,476, filed on Aug. 21, 1998.
(60) Provisional application No. 60/113,212, filed on Dec. 22, 1998.
(60) Provisional application No. 60/119,328, filed on Feb. 9, 1999.
(60) Provisional application No. 60/135,585, filed on May 24, 1999.
(60) Provisional application No. 60/135,586, filed on May 24, 1999.

(51) Int. Cl. A61K 31/53; A61K 31/501; A61K 31/454; A61K 31/4439; A61K 31/427; A61K 31/421; A61K 31/4162

(52) U.S. Cl. 514/241; 514/369; 514/252.05; 514/430; 514/431; 514/362; 514/376; 514/364; 514/381; 514/386

(57) ABSTRACT

Compounds, compositions and methods are provided for the treatment and prophylaxis of infections and associated diseases caused by viruses of the Flaviviridae family by administering certain rhodanine derivatives, and analogs thereof, tri- and tetracyclic rhodanine alkanoic acids and rhodanine benzoic acids being particularly effective.

This application claims the benefit of the following U.S. Provisional Applications: No. 60/097,476, filed Aug. 21, 1998; No. 60/113,212, filed Dec. 22, 1998; No. 60/119,328, filed Feb. 9, 1999; No. 60/135,585, filed May 24, 1999 and No. 60/135,586, filed May 24, 1999. The entire disclosure of each of the aforesaid Provisional Applications is incorporated by reference in the present application, as though set forth in full.

### FIELD OF THE INVENTION

The present invention relates to novel rhodanine derivatives and analogs, as well as compositions containing the same and to the use thereof for treating or preventing viral infections and diseases associated therewith, particularly those viral infections and associated diseases caused by viruses within the Flaviviridae family.

### BACKGROUND OF THE INVENTION

The Flaviviridae family consists of three genera and several viruses that are currently unassigned to specific genera. The hepacivirus genus includes the hepatitis C viruses (HCV). Viruses such as GB virus-A and GB virus-A-like agents, GB virus-B and GBV-C or hepatitis G virus, while at present not formally classified within the hepacivirus genus, are closely related to HCV and represent unassigned members of the Flaviviridae family. Also within the Flaviviridae is the pestivirus genus, which includes bovine viral diarrhea viruses (BVDV), border disease viruses and classical swine fever virus, and the flavivirus genus, with viruses such as dengue, yellow fever, Japanese encephalitis and tick-borne encephalitis viruses.

Viruses within this family cause significant disease in human and animal populations. HCV is a major cause of human hepatitis globally. The World Health Organization estimates that 170 million people worldwide are presently infected with the virus. Most infections become persistent and about 60% of cases develop chronic liver disease. Chronic HCV infection can lead to development of cirrhosis, hepatocellular carcinoma and liver failure.

Interferon and interferon in combination with ribavirin are used in the U.S. for hepatitis due to HCV. These treatments are associated with improved serum enzyme response in some patients. The remainder are non-responsive to treatment. For responders, a sustained clinical improvement is seen in only a small percentage of patients; the majority of patients relapse upon cessation of treatment. Thus, the effectiveness of therapy for chronic hepatitis C is variable and its cure rate remains low. Moreover, therapy is often associated with considerable side effects.

Pestivirus infections of domesticated livestock cause significant economic losses worldwide. Pestiviruses cause a range of clinical manifestations including abortion, teratogenesis, respiratory problems, chronic wasting disease, immune system dysfunction and predisposition to secondary viral and bacterial infections. Certain BVDV strains cause an acute fatal disease. BVDV can also establish persistent infections in fetuses. When born, these persistently infected (PI) animals remain viremic throughout life and serve as continuous virus reservoirs. PI animals often succumb to fatal mucosal disease.

Flaviviruses are important pathogens of man and are also prevalent throughout the world. There are at least 38 flaviviruses associated with human disease, including the dengue fever viruses, yellow fever virus and Japanese encephalititis virus. Flaviviruses cause a range of acute febrile illnesses and encephalitic and hemorrhagic diseases.

Currently, there are no antiviral pharmaceuticals to prevent or treat pestivirus or flavivirus infections.

New therapies and preventatives are clearly needed for infections and diseases caused by viruses of Flaviviridae family.

In considering approaches to the diagnosis, control, prevention and treatment of infections and associated diseases caused by viruses, it is often desirable to identify virus-specific functions that may be exploited in such approaches. In particular, enzymatic activities of virus-encoded polypeptides are quite useful. These virus-specified components are often essential for virus replication and may be suitable targets for antiviral drug discovery strategies.

One such target that plays a central role in the life cycle of many RNA viruses is the virus-encoded RNA-dependent RNA polymerase (RdRp) protein. Regarding viruses of the Flaviviridae, this protein is termed NS5B in the case of the hepaciviruses and pestiviruses, and NS5 in the case of the flaviviruses (collectively referred to as NS5). RdRp proteins are a key component of the virus replicase complex, enabling the virus to replicate its RNA genome and produce progeny viruses. The RdRp of RNA viruses is an attractive target for antiviral drug development.

### SUMMARY OF THE INVENTION

According to one aspect of the invention, there is provided a method of treating or preventing infection caused by at least one virus of the Flaviviridae and disease associated with such infection in a living host having or susceptible to such infection. The method comprises administering to the infected or susceptible host a therapeutically or prophylactically effective amount of a compound, or precursor of said compound, having the formula:

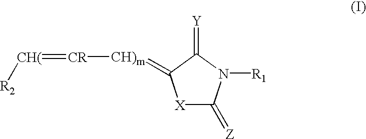

wherein

R represents hydrogen or alkyl; and m is an integer from 0-4;

R₁ represents hydrogen or a radical selected from those consisting of an —R₃COOH radical, wherein R₃ is an unsubstituted or substituted, branched or straight chain, saturated or unsaturated hydrocarbon moiety of 1-10 carbon atoms, an unsubstituted or substituted phenyl (C₆H₅) radical or an unsubstituted or substituted phenylalkyl radical, the R₃ substituents being at least one selected from those consisting of a branched or straight chain, saturated or unsaturated aliphatic group having 1-6 carbon atoms, an unsubstituted or substituted heterocyclic radical or an unsubstituted or substituted phenyl (C₆H₅) radical, said heterocyclic radical being selected from those consisting of furan, thiophene, oxazole, oxadiazole, pyridine, pyrimidine, pyrazole, triazole, pyridazine, 1,3-oxathiolane, thiazole, thiadiazole, imidazole, tetrazole, pyrrole and triazine;

X represents a moiety selected from the group consisting of —S—, —O— or —N(Rₐ)—, Rₐ being hydrogen or alkyl of 1-5 carbon atoms;

R₂ represents a radical selected from those consisting of an unsubstituted or substituted phenylalkyl radical, an unsubstituted or substituted phenylalkenyl radical, an unsubstituted or substituted phenylalkynyl radical, an unsubstituted or substituted biphenylalkyl radical, an unsubstituted or substituted polycyclic radical, an unsubstituted or substituted alicyclic radical having 5-8 carbon atoms or a radical of the formula (R₂ₐ—)ₙ(L—)ₚR_{2b}—, wherein R₂ₐ and R_{2b} may be the same or different and represent an unsubstituted or substituted heterocyclic radical or an unsubstituted or substituted phenyl radical, R₂ₐ also represents an unsubstituted or substituted polycyclic radical and L represents a divalent linking moiety selected from the group consisting of a valence bond, —(CH₂)_{q}—, —HC═CH—, —C═C—, —C(═O)—, —O—, —S—, —S(═O)—, —S(═O)₂— or NR_{2c}, R_{2c}. being hydrogen or alkyl, n and p are each 0 or 1, and q is an integer from 1 to 3;

said heterocyclic radicals being selected from the group consisting of furan, thiophene, oxazole, oxadiazole, isoxazole, pyridine, pyrimidine, pyrazole, triazole, pyridazine, 1,3-oxathiolane, thiazole, isothiazole, thiadiazole, imidazole, pyrrole, tetrazole and triazine;

said polycyclic radicals being selected from the group consisting of benzofuran, isobenzofuran, benzothiophene, isobenzothiophene, benzoxazole, indole, 2-isoindole, benzopyrazole, quinoline, isoquinoline, 1,2-benzodiazine, 1,3-benzodiazine, 1,2,3-benzotriazole, benzothiazole, benzimidazole, 1,2,3-benzotriazine, 1,2,4-benzotriazine, naphthalene, anthracene and fluorene;

the heterocyclic radical substituents, the polycyclic radical substituents and the alicyclic radical substituents being at least one selected from the group consisting of a straight or branched chain, saturated or unsaturated aliphatic group having 1-6 carbon atoms, halogen, perhaloalkyl, monohaloalkyl, dihaloalkyl, alkoxy, acyl, acyloxy, acyloxyalkyl, phenylalkoxy, hydroxy, hydroxyalkyl, thio, alkylthio, nitro, carboxy, carbalkoxy;

the phenyl radical substituents, the phenylalkyl radical substituents, the phenylalkenyl radical substituents, the phenylalkynyl radical substituents and the biphenylalkyl radical substituents being at least one selected from the group consisting of a straight or branched chain, saturated or unsaturated aliphatic group having 1-6 carbon atoms, halogen, nitro, carboxy, hydroxy, hydroxyalkyl, perhaloalkyl, monohaloalkyl, dihaloalkyl, alkoxy, phenylalkoxy, acyl, acyloxy, acyloxyalkyl, cyano, carbalkoxy, thio, alkylthio, alkylsulfinyl, alkylsulfonyl, amino, alkylamino, dialkylamino, aminoalkyl, alkylaminoalkyl, dialkylaminoalkyl, sulfonamido, carboxamido, alkanoylamino;

Y represents O or S;

Z represents O, S or N(R_{b}), R_{b} being hydrogen or alkyl; or R₁ and R_{b} may be joined to form an imidazole or a benzimidazole moiety; and the isomers and pharmaceutically acceptable salts of the compound.

Infections caused by Flaviviridae viruses and associated diseases may be effectively treated or prevented by administering a compound of the formula:

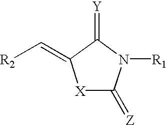

wherein

R₁ represents hydrogen or a radical selected from those consisting of —R₃COOH, wherein R₃ is a branched or straight chain aliphatic moiety of 1-10 carbon atoms, or an unsubstituted, or substituted phenyl (C₆H₅) group;

X represents a moiety selected from the group consisting of —S—, 13 O— or —N(Rₐ)—, Rₐ being hydrogen or alkyl of 1-5 carbon atoms;

R₂ represents a radical selected from those consisting of an unsubstituted or substituted heterocyclic group, an unsubstituted or substituted bicyclic ring moiety, an unsubstituted or substituted phenyl group, an unsubstituted or substituted biphenyl (C₆H₅—C₆H₄) group or an unsubstituted or substituted cinnamenyl (C₆H₅CH═CH—) group, the heterocyclic group being selected from those consisting of furan, thiophene, oxadiazole, pyridine, pyrimidine, pyrazole, triazole, pyridazine, 1,3-oxathiolane, thiazole, thiadiazole, imidazole, pyrrole and triazine, said bicyclic ring moiety being selected from those consisting of benzofuran, isobenzofuran, benzothiophene, isobenzothiophene, benzoxazole, benzopyrrole, indolenine, 2-isobenzazole, benzpyrazole, quinoline, isoquinoline, 1,2-benzodiazine, 1,3-benzodiazine, 1,2,3-benzotriazole, benzothiazole, benzimidazole, 1,2,3-benzotriazine and 1,2,4-benzotriazine, the heterocyclic group and bicyclic ring moiety substituents being at least one selected from those consisting of alkyl of 1-5 carbon atoms, halogen, alkoxy, hydroxy, nitro or an unsubstituted or substituted phenyl group;

the phenyl group substituents, the biphenyl group substituents and the cinnamenyl group substituents being at least one selected from those consisting of halogen, nitro, carboxy, hydroxy, alkyl of 1-5 carbon atoms, trifluoromethyl, alkoxy, acyloxy, cyano, carbalkoxy, alkylthio, alkylsulfinyl, alkylsulfonyl, amino, alkylamino, dialkylamino, sulfonamido or carboxamido;

Y represents O or S;

Z represents O, S or N(R_{b}), R_{b} being hydrogen or alkyl of 1-5 carbon atoms;

or R₁ and R_{b} may be joined to form a benzimidazole moiety; and the isomers and pharmaceutically acceptable salts of the compound.

According to another aspect of this invention, pharmaceutical compositions for treating or preventing viral infections are provided, which comprise an anti-viral agent in an amount effective to attenuate viral infectivity, and a pharmaceutically acceptable carrier medium. In one embodiment, the composition of the invention comprises a compound of the formula:

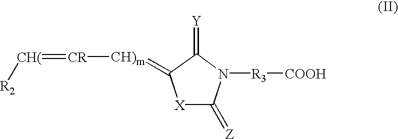

wherein

R represents hydrogen or alkyl; and m is an integer from 0-4;

R₃ represents an unsubstituted or substituted, branched or straight chain, saturated or unsaturated hydrocarbon moiety having 1-10 carbon atoms in the main chain, the hydrocarbon moiety substituents being at least one selected from those consisting of a branched or straight chain, saturated or unsaturated aliphatic group, having 1-6 carbon atoms, an unsubstituted or substituted heterocyclic radical or an unsubstituted or substituted phenyl (C₆H₅) radical, the heterocyclic radical being selected from those consisting of furan, thiophene, oxazole, oxadiazole, pyridine, pyrimidine, pyrazole, triazole, pyridazine, 1,3-oxathiolane, thiazole, thiadiazole, imidazole, tetrazole, pyrrole and triazine; and R₂, X, Y, Z and the substituents of the heterocyclic radicals, the polycyclic radicals, the alicyclic radicals, the phenyl radicals, the phenylalkyl radicals, the phenylalkenyl radicals, the phenylalkynyl radicals and the biphenylalkyl radicals are as previously defined relative to formula I, above. According to this embodiment, the anti-viral agent may comprise a compound of the formula:

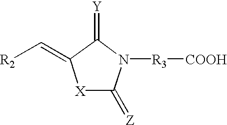

wherein

R₃ represents an unsubstituted or substituted, branched or straight chain, saturated or unsaturated aliphatic moiety having 1-10 carbon atoms in the main chain, the aliphatic moiety substituents being selected from those consisting of at least one branched or straight chain, saturated or unsaturated aliphatic group, having 1-6 carbon atoms, unsubstituted or substituted mono-heterocyclic group or unsubstituted or substituted phenyl (C₆H₅) group, the heterocyclic group being selected from those consisting of furan, thiophene, oxazole, oxadiazole, pyridine, pyrimidine, pyrazole, triazole, pyridazine, 1,3-oxathiolane, thiazole, thiadiazole, imidazole, tetrazole, pyrrole and triazine.

X represents a moiety selected from the group consisting of —S—, —O— or —N(Rₐ)—, Rₐ being hydrogen or alkyl;

R₂ represents a radical selected from those consisting of an unsubstituted or substituted mono- or bi-heterocyclic group, an unsubstituted or substituted polycyclic ring moiety, an unsubstituted or substituted alicyclic group having 5-8 carbon atoms, an unsubstituted or substituted phenyl group, an unsubstituted or substituted biphenyl (C₆H₅—C₆H₄—) group, an unsubstituted or substituted phenyl ether group (C₆H₅—O—C₆H₄—) or an unsubstituted or substituted cinnamenyl (C₆H₅CH═CH—) group, the mono-heterocyclic group being selected from those consisting of furan, thiophene, oxazole, oxadiazole, pyridine, pyrimidine, pyrazole, triazole, pyridazine, 1,3-oxathiolane, thiazole, thiadiazole, imidazole, tetrazole, pyrrole and triazine, the bi-heterocyclic group comprising two heterocyclic groups which are selected from said mono-heterocyclic group members, and which may be the same or different, said polycyclic ring moiety being selected from those consisting of benzofuran, isobenzofuran, benzothiophene, isobenzothiophene, benzoxazole, benzopyrrole, indolenine, 2-isobenzazole, benzpyrazole, quinoline, isoquinoline, 1,2-benzodiazine, 1,3-benzodiazine, 1,2,3-benzotriazole, benzothiazole, benzimidazole, 1,2,3-benzotriazine and 1,2,4-benzotriazine, naphthalene, anthracene and fluorene;

the mono- or bi-heterocyclic group substituents, the alicyclic group substituents and the polycyclic ring moiety substituents being at least one selected from those consisting of a straight or branched chain, saturated or unsaturated aliphatic group having 1-6 carbon atoms, halogen, trifluoromethyl, alkoxy, hydroxy, thio, nitro, an unsubstituted or substituted phenyl group, an unsubstituted or substituted phenylalkenyl group or an unsubstituted or substituted phenylalkynyl group;

the phenyl group substituents, the biphenyl group substituents, the phenyl ether group substituents, the phenytalkenyl group substituents, the phenylalkynyl group substituents and the cinnamenyl group substituents being at least one selected from those consisting of a straight or branched chain, saturated or unsaturated aliphatic group having 1-6 carbon atoms, halogen, nitro, carboxy, hydroxy, trifluoromethyl, difluoromethyl, alkoxy, phenoxy, acyloxy, cyano, carbalkoxy, thio, alkylthio, alkylsulfinyl, alkylsulfonyl, amino, alkylamino, dialkylamino, sulfonamido, carboxamido, alkanoylamino, 1-pyrrolidyl, 1-piperidinyl or 4-morpholinyl;

Y represents O or S;

Z represents O, S or N(R_{b}), R_{b} being hydrogen or alkyl;

or R₁ and R_{b} may be joined to form an imidazole or benzimidazole moiety; and the isomers and pharmaceutically acceptable salts of such compound.

In another embodiment, the composition of the invention comprises a compound of the formula:

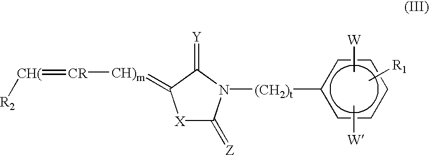

wherein

R represents hydrogen or alkyl; and m is an integer from 0-4;

R₁ represents hydrogen or a substituent selected from the group consisting of —OH, —COOR₄, —CONR₅R₆, —SO₂NR₇R₈, R₄, R₅, R₆, R₇ and R₈ being independently selected from the group of hydrogen or alkyl, or R₁ represents a mono-heterocyclic radical selected from the group of furan, thiophene, pyridine, pyrimidine, pyridazine, 1,3-oxathiolane, tetrazole, oxadiazole, oxazole, triazole, imidazoline, imidazole, thiazole, thiadiazole, pyrrole, piperidine, morpholine, triazine and pyrazole;

W and W′ may be the same or different and represent hydrogen or a substituent selected from the group consisting of a straight or branched chain, saturated or unsaturated aliphatic group having 1-6 carbon atoms, halogen, nitro, hydroxy, perfluoroalkyl, difluoromethyl, alkoxy, phenoxy, phenylalkoxy, acyl, acyloxy, acyloxyalkyl, cyano, carbalkoxy, thio, alkylthio, alkylsulfinyl, alkylsulfonyl, amino, alkylamino, dialkylamino, sulfonamido, carboxamido and alkanoylamino;

t is an integer from 0 to 8; and R₂, X, Y, Z and the substituents of the heterocyclic radicals, the polycyclic radicals, the alicyclic radicals, the phenyl radicals, the phenylalkyl radicals, the phenylalkenyl radicals, the phenylalkynyl radicals and the biphenylalkyl radicals are as previously defined relative to formula I, above. According to this embodiment, the anti-viral agent may comprise a compound of the formula:

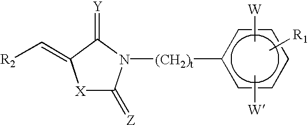

wherein

R₁ represents hydrogen or a substituent selected from the group consisting of —OH, —COOR₃, —CONR₄R₅, —SO₂NR₆R₇, R₃, R₄, R₅, R₆ and R₇ independently selected from the group of hydrogen, alkyl, or R₁ represents a heterocylic ring selected from the group of tetrazole, oxadiazole, oxazole, triazole, imidazoline, imidazole, thiazole, thiadiazole, pyrrole, piperidine, morpholine and pyrazole;

W and W′ may be the same or different and represent hydrogen or a substituent selected from the group consisting of a straight or branched chain, saturated or unsaturated aliphatic group having 1-6 carbon atoms, halogen, nitro, hydroxy, perfluoroalkyl, difluoromethyl, alkoxy, phenoxy, acyloxy, cyano, carbalkoxy, thio, alkylthio, alkylsulfinyl, alkylsulfonyl, amino, alkylamino, dialkylamino, sulfonamide, carboxamide and alkanoylamino.

t is an integer from 0 to 8;

X represents a moiety selected from the group consisting of —S—, —O— or —N(R_{a)—, R}ₐ being hydrogen or alkyl;

R₂ represents a radical selected from those consisting of an unsubstituted or substituted mono- or bi-heterocyclic group, an unsubstituted or substituted polycyclic ring moiety, an unsubstituted or substituted alicyclic group having 5-8 carbon atoms, an unsubstituted or substituted phenyl group, an unsubstituted or substituted biphenyl (C₆H₅—C₆H₄—) group, an unsubstituted or substituted phenyl ether group (C₆H₅—O—C₆H₄—), an unsubstituted or substituted cinnamenyl (C₆H₅CH═CH—) group, or an unsubstituted or substituted stilbenyl group, said mono-heterocyclic group being selected from those consisting of furan, thiophene, oxazole, oxadiazole, pyridine, pyrimidine, pyrazole, triazole, pyridazine, 1,3-oxathiolane, thiazole, thiadiazole, imidazole, tetrazole, pyrrole and triazine, said bi-heterocyclic group comprising two heterocyclic groups, said two heterocyclic groups being selected from said mono-heterocyclic groups and being the same or different, said polycyclic ring moiety being selected from those consisting of benzofuran, isobenzofuran, benzothiophene, isobenzothiophene, benzoxazole, benzopyrrole, indolenine, 2-isobenzazole, benzpyrazole, quinoline, isoquinoline, 1,2-benzodiazine, 1,3-benzodiazine, 1,2,3-benzotriazole, benzothiazole, benzimidazole, 1,2,3-benzotriazine, 1,2,4-benzotriazine, naphthalene, anthracene and fluorene;

the mono-heterocyclic group substituents, the bi-heterocyclic group substituents, the alicyclic group substituents and the polycyclic ring moiety substituents being at least one selected from those consisting of a straight or branched chain, saturated or unsaturated aliphatic group having 1-6 carbon atoms, halogen, trifluoromethyl, alkoxy, hydroxy, thio, nitro, carboxy, carbalkoxy, an unsubstituted or substituted phenyl group, an unsubstituted or substituted phenylalkyl group, an unsubstituted or substituted phenylalkenyl group or an unsubstituted or substituted phenylalkynyl group;

the phenyl group substituents, the biphenyl group substituents, the phenyl ether group substituents, the phenylalkyl group substituents, the phenylalkenyl group substituents, the phenylalkynyl group substituents, the cinnamenyl group substituents and the stilbenyl group substituents being at least one selected from those consisting of a straight or branched chain, saturated or unsaturated aliphatic group having 1-6 carbon atoms, halogen, nitro, carboxy, hydroxy, trifluoromethyl, difluoromethyl, alkoxy, phenoxy, acyloxy, cyano, carbalkoxy, thio, alkylthio, alkylsulfinyl, alkylsulfonyl, amino, alkylamino, dialkylamino, sulfonamido, carboxamido, alkanoylamino, 1-pyrrolidyl, 1-piperidinyl or 4-morpholinyl;

Y represents O or S;

Z represents O, S or N(R_{b}), R_{b} being hydrogen or alkyl;

or R₁ and R_{b} may be joined to form an imidazole or benzimidazole moiety; and the isomers and pharmaceutically acceptable salts of the compound.

Preferably the R₂ radical in formulas II and III, above, is of the formula (R₂ₐ—)ₙ(L—)ₚR_{2b}—, p is 0; and m is 0.

According to a further aspect of this invention, compounds are provided which have the formula:

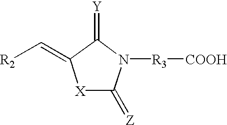

wherein

R₃ represents an unsubstituted or substituted, branched or straight chain, saturated or unsaturated hydrocarbon moiety having 1-10 carbon atoms in the main chain, the hydrocarbon moiety substituents being at least one selected from those consisting of a branched or straight chain, saturated or unsaturated aliphatic group, having 1-6 carbon atoms, unsubstituted or substituted mono-heterocyclic group or unsubstituted or substituted phenyl (C₆H₅) group, the mono-heterocyclic group being selected from those consisting of furan, thiophene, oxazole, oxadiazole, pyridine, pyrimidine, pyrazole, triazole, pyridazine, 1,3-oxathiolane, thiazole, thiadiazole, imidazole, tetrazole, pyrrole and triazine;

X represents a moiety selected from the group consisting of —S—, —O— or —N(Rₐ)—, Rₐ being hydrogen or alkyl;

R₂ represents a radical selected from those consisting of an unsubstituted or substituted mono- or bi-heterocyclic radical, an unsubstituted or substituted polycyclic radical, an unsubstituted or substituted polycyclic-heterocyclic radical, an unsubstituted or substituted alicyclic radical having 5-8 carbon atoms, an unsubstituted or substituted phenyl radical, an unsubstituted or substituted biphenyl (C₆H₅—C₆H₄—) radical, an unsubstituted or substituted phenyl ether (C₆H₅—O—C₆H₄—) radical or an unsubstituted or substituted 2-phenylethenyl (C₆H₅CH═CH—) radical, said mono-heterocyclic group being selected from those consisting of furan, thiophene, oxazole, oxadiazole, pyridine, pyrimidine, pyrazole, triazole, pyridazine, 1,3-oxathiolane, thiazole, thiadiazole, imidazole, tetrazole, pyrrole and triazine, said bi-heterocyclic group comprising two heterocyclic moieties which are selected from the mono-heterocyclic radical group members, and which may be the same or different, said polycyclic ring moiety being selected from those consisting of benzofuran, isobenzofuran, benzothiophene, isobenzothiophene, benzoxazole, indole, 2-isoindole, benzopyrazole, quinoline, isoquinoline, 1,2-benzodiazine, 1,3-benzodiazine, 1,2,3-benzotriazole, benzothiazole, benzimidazole, 1,2,3-benzotriazine and 1,2,4-benzotriazine, naphthalene, anthracene and fluorene and said polycyclic-heterocyclic radical comprising a polycyclic moiety selected from said polycyclic radical group members and a heterocyclic moiety which is selected from the mono-heterocyclic radical group members;

the mono-heterocyclic radical substituents, the bi-heterocyclic radical substituents, the alicyclic radical substituents, the polycyclic radical substituents and the polycyclic-heterocyclic radical substituents being at least one selected from those consisting of a straight or branched chain, saturated or unsaturated aliphatic group having 1-6 carbon atoms, halogen, trifluoromethyl, alkoxy, hydroxy, thio, nitro, carbalkoxy, an unsubstituted or substituted phenyl radical, an unsubstituted or substituted phenylalkenyl radical or an unsubstituted or substituted phenylalkynyl radical;

the phenyl radical substituents, the biphenyl radical substituents, the phenyl ether radical substituents, the phenylalkenyl radical substituents, the phenylalkynyl radical substituents and the 2-phenylethenyl radical substituents being at least one selected from those consisting of a straight or branched chain, saturated or unsaturated aliphatic group having 1-6 carbon atoms, halogen, nitro, carboxy, hydroxy, trifluoromethyl, difluoromethyl, alkoxy, phenoxy, acyloxy, cyano, carbalkoxy, thio, alkylthio, alkylsulfinyl, alkylsulfonyl, amino, alkylamino, dialkylamino, sulfonamido, carboxamido, alkanoylamino, 1-pyrrolidyl, 1-piperidinyl or 4-morpholinyl;

Y represents O or S;

Z represents O, S or N(R_{b}), R_{b} being hydrogen or alkyl;

or R₁ and R_{b} may be joined to form an imidazole or benzimidazole moiety; and the isomers and pharmaceutically acceptable salts of the compound.

According to still another aspect of this invention, compounds are provided which have the formula:

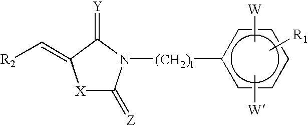

wherein

R₁ represents hydrogen or a substituent selected from the group consisting of —OH, —COOH, —CONR₄R₅, —SO₂NR₆R₇, R₄, R₅, R⁶ and R₇ being independently selected from the group of hydrogen, alkyl, or R₁ represents a heterocylic ring selected from the group of furan, thiophene, pyridine, pyrimidine, pyridazine, 1,3-oxathiolane, tetrazole, oxadiazole, oxazole, triazole, imidazoline, imidazole, thiazole, thiadiazole, pyrrole, piperidine, morpholine, triazine and pyrazole;

W and W′ may be the same or different and represent hydrogen or a substituent selected from the group consisting of a straight or branched chain, saturated or unsaturated aliphatic group having 1-6 carbon atoms, halogen, nitro, hydroxy, perfluoroalkyl, difluoromethyl, alkoxy, phenoxy, acyloxy, cyano, carbalkoxy, thio, alkylthio, alkylsulfinyl, alkylsulfonyl, amino, alkylamino, dialkylamino, sulfonamido, carboxamido and alkanoylamino.

t is an integer from 0 to 8;

X represents a moiety selected from the group consisting of —S—, —O— or —N(Rₐ)—, Rₐ being hydrogen or alkyl;

R₂ represents a radical selected from those consisting of an unsubstituted or substituted mono- or bi-heterocyclic radical, an unsubstituted or substituted polycyclic radical, an unsubstituted or substititued polycyclic-heterocyclic radical, an unsubstituted or substituted alicyclic radical having 5-8 carbon atoms, an unsubstituted or substituted phenyl radical, an unsubstituted or substituted biphenyl (C₆H₅—C₆H₄—) radical, an unsubstituted or substituted phenyl ether (C₆H₅—O—C₆H₄—) radical, an unsubstituted or substituted 2-phenylethenyl (C₆H₅CH═CH—) radical, or an unsubstituted or substituted stilbenyl (C₆H₅—CH═CH—C₆H₄—) radical, the mono-heterocyclic radical being selected from those consisting of furan, thiophene, oxazole, oxadiazole, pyridine, pyrimidine, pyrazole, triazole, pyridazine, 1,3-oxathiolane, thiazole, thiadiazole, imidazole, tetrazole, pyrrole and triazine; the bi-heterocyclic group comprising two heterocyclic groups, the two heterocyclic groups being selected from said mono-heterocyclic radical group members and being the same or different, the polycyclic radical being selected from the group consisting of benzofuran, isobenzofuran, benzothiophene, isobenzothiophene, benzoxazole, benzopyrrole, 2-isoindole, benzopyrazole, quinoline, isoquinoline, 1,2-benzodiazine, 1,3-benzodiazine, 1,2,3-benzotriazole, benzothiazole, benzimidazole, 1,2,3-benzotriazine, 1,2,4-benzotriazine, naphthalene, anthracene and fluorene, and the polycyclic-heterocyclic radical comprising a polycyclic moiety selected from the polycyclic radical group members and a heterocyclic moiety selected from the mono-heterocyclic radical group members;

the mono-heterocyclic radical substituents, the bi-heterocyclic radical substituents, the alicyclic radical substituents, the polycyclic radical substituents and the polycyclic-heterocyclic radical substituents being at least one selected from those consisting of a straight or branched chain, saturated or unsaturated aliphatic group having 1-6 carbon atoms, halogen, trifluoromethyl, alkoxy, hydroxy, thio, nitro, acyl, carboxy, carbalkoxy, an unsubstituted or substituted phenyl radical, an unsubstituted or substituted phenylalkyl radical, an unsubstituted or substituted phenylalkenyl radical or an unsubstituted or substituted phenylalkynyl radical;

the phenyl radical substituents, the biphenyl radical substituents, the phenyl ether radical substituents, the phenylalkyl radical substituents, the phenylalkenyl radical substituents, the phenylalkynyl radical substituents, the 2-phenylethenyl radical substituents and the stilbenyl radical substituents being at least one selected from those consisting of a straight or branched chain, saturated or unsaturated aliphatic group having 1-6 carbon atoms, halogen, nitro, carboxy, hydroxy, trifluoromethyl, difluoromethyl, alkoxy, phenoxy, phenylalkoxy, acyl, acyloxy, cyano, carbalkoxy, thio, alkylthio, alkylsulfinyl, alkylsulfonyl, amino, alkylamino, dialkylamino, sulfonamido, carboxamido, alkanoylamino, furan, thiophene, pyridine, pyrimidine, pyridazine, 1,3-oxathiolane, tetrazole, oxadiazole, oxazole, triazole, imidazoline, imidazole, thiazole, thiadiazole, pyrrole, piperidine, morpholine and pyrazole;

Y represents O or S;

Z represents O, S or N(R_{b}), R_{b} being hydrogen or alkyl;

or R₁ and R_{b} may be joined to form an imidazole or benzimidazole moiety; and the isomers and pharmaceutically acceptable salts of the compound.

### DETAILED DESCRIPTION OF THE INVENTION

Rhodanine derivatives or analogs according to the present invention can be conveniently prepared from known starting materials by following the general synthetic scheme shown below.

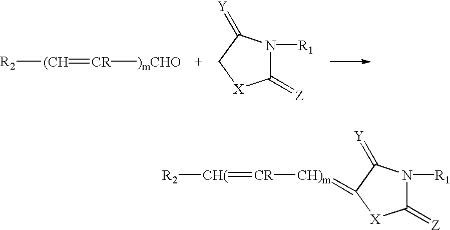

wherein R, R₁, R₂, X, Y, Z and m are as previously defined.

In carrying out the general synthetic scheme illustrated above, a reaction mixture is prepared, which comprises the appropriate aldehyde and the appropriate rhodanine derivative or analog in ethanol, and the reaction mixture is heated to reflux in the presence of a catalytic amount of piperidine. The appropriate aldehyde starting materials or precursors thereof are available from commercial sources. Furthermore, various 5-substituted furaldehydes can be prepared by treating the corresponding dimethylacetal as shown below. Specifically, 5-bromofuran-2-carboxaldehyde dimethylacetal is treated with n-butyl lithium and n-tributyltin chloride in tetrahydrofuran at −78° C. to produce the tri-n-butylfuran analog which, on treatment with the appropriate substituted bromobenzene, yields the 5-substituted furan intermediate. Conversion of the resulting intermediate with pyridine, using a catalytic amount of pyridinium p-toluene sulfonate (PPTS), provides a 5-(substituted phenyl) furan-2-carboxaldehyde.

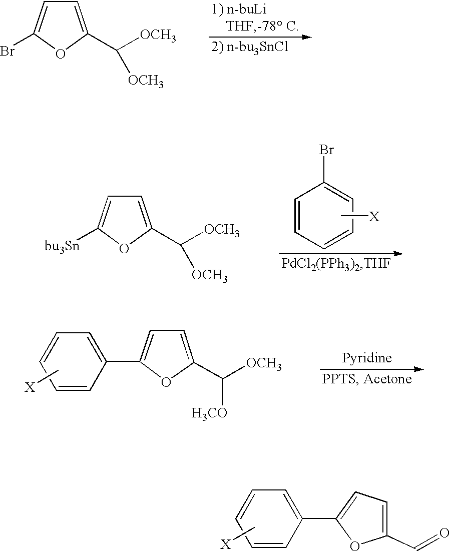

The aldehydes may also be prepared by the method described by Pong et al., Arzneim. Forsch., 33: 1411 (1983).

All possible isomers of formulas I-III, above, are within the scope of the present invention. Representative examples of such isomers include, without limitation, the E and Z isomers, as well as the various isomers of heterocyclic substituents that may be present in the compounds of the present invention.

In vitro studies have been performed which demonstrate the usefulness of compounds described herein as antiviral agents. Antiviral activity was measured by the inhibitory activity of the compounds against the viral RdRp in an enzymological assay for RNA synthesis.

Among the preferred compounds for practicing this invention are compounds of formula II, above, wherein R₃ is a straight chain alkylene of 1-5 carbon atoms, Y is oxygen, X and Z are sulfur and R₂ is an unsubstituted or substituted mono-heterocyclic radical selected from those consisting of furan, thiophene and oxazole, or an unsubstituted or substituted bi-heterocyclic radical selected from those consisting of bi-thienyl and 1H-pyrazolylthienyl, the heterocyclic radical substituents being at least one selected from those consisting of halogen, trifluoromethyl or an unsubstituted or substituted phenyl radical, and said phenyl radical substituents being at least one selected from those consisting of halogen, nitro, carboxy, hydroxy,methyl, ethyl, trifluoromethyl, difluoromethyl, alkoxy, phenoxy, acyloxy, cyano, carbalkoxy, thio, alkylthio, alkylsulfinyl, alkylsulfonyl, amino, alkylamino, dialkylamino, sulfonamido, carboxamido, alkanoylamino, 1-pyrrolidinyl, 1-piperidinyl or 4-morpholinyl.

Additional preferred compounds are those of formula II, above, wherein R₃ is a straight chain alkylene of 1-5 carbon atoms, Y is oxygen, X and Z are sulfur and R₂ is an unsubstituted or substituted phenyl radical, the phenyl radical substituents being at least one selected from those consisting of halogen, nitro, carboxy, hydroxy,methyl, ethyl, trifluoromethyl, difluoromethyl, alkoxy, phenoxy, acyloxy, cyano, carbalkoxy, thio, alkylthio, alkylsulfinyl, alkylsulfonyl, amino, alkylamino, dialkylamino, sulfonamido, carboxamido, alkanoylamino, 1-pyrrolidinyl, 1-piperidinyl or 4-morpholinyl.

Also preferred are compounds of formula II above, wherein R₃ is a straight chain alkylene of 1-5 carbon atoms, Y is oxygen, X and Z are sulfur and R₂ is an unsubstituted or substituted polycyclic radical selected from those consisting of 9-phenanthryl and 2-fluorenyl, said polycyclic radical substituents being at least one selected from those consisting of methyl, ethyl, halogen, alkoxy, hydroxy, thio, nitro or an unsubstituted or substituted phenyl radical, the phenyl radical substituents being at least one selected from those consisting of halogen, nitro, carboxy, hydroxy,methyl, ethyl, trifluoromethyl, difluoromethyl, alkoxy, phenoxy, acyloxy, cyano, carbalkoxy, thio, alkylthio, alkylsulfinyl, alkylsulfonyl, amino, alkylamino, dialkylamino, sulfonamido, carboxamido, alkanoylamino, 1-pyrolidyl, 1-piperidinyl or 4-morpholinyl.

Preferred among the compounds of formula III, above, are those wherein R₁ is a carboxyl group, W and W′ represent hydrogen, halogen, hydroxy, alkyl or trifluoromethyl substituents, Y is oxygen, X and Z are sulfur, R₂ represents an unsubstituted or substituted furan group or an unsubstituted or substituted thiophene group, the furan substituent(s) and the thiophene substituent(s) being at least one selected from those consisting of alkyl, monohalophenyl, dihalophenyl, monohalocarboxyphenyl, carboxyphenyl, trifluoromethylphenyl, monohalotrifluoromethylphenyl, phenylethynyl, monoalkylphenyl, dialkylphenyl, furanyl, and thienyl, m=0 and t=0.

R₂ in the compounds of formulas II and III, above, is also preferably an unsubstituted or substituted thiazole, the thiazole substituents being the same as the furan and thiophene substituents in the next preceding paragraph.

Other preferred compounds for practicing this invention are those of formula III, above, wherein R₁ is a carboxyl group, W and W′ represent hydrogen, halogen, hydroxy or trifluoromethyl substituents, Y is oxygen, X and Z are sulfur, R₂ represents an unsubstituted or substituted phenyl group, the phenyl substituent(s) being at least one selected from those consisting of halogen, alkoxy, carboxy, an unsubstituted or substituted 2-phenylethenyl group, an unsubstituted or substituted furan group, or an unsubstituted or substituted thiophene group, the 2-phenylethenyl substituent(s), the furan substituent(s) and the thiophene substituent(s) being at least one selected from those consisting of a straight or branched chain, saturated or unsaturated aliphatic group having 1-6 carbon atoms, halogen, nitro, carboxy, hydroxy, trifluoromethyl, difluoromethyl, alkoxy, phenoxy, phenylalkoxy, acyloxy, cyano, carbalkoxy, thio, alkylthio, alkylsulfinyl, alkylsulfonyl, amino, alkylamino, dialkylamino, sulfonamide, carboxamide or alkanoylamino, m=0 and t=0. In the compounds of formula III, W and W′ also preferably represent methyl (CH₃) groups.

The term “alkyl” as used herein refers to aliphatic hydrocarbon radicals of one to six carbon atoms in length. Similarly, the term “alkyl”, or any variation thereof, used in combination form to name substituents, such as alkoxy, alkylthio, alkylamino, alkylsulfinyl or alkylsulfonyl also refers to aliphatic hydrocarbon radicals of one to six carbon atoms in length.

The term “acyl” is used herein in accordance with its ordinary meaning to refer to an organic radical derived from a carboxylic acid by the removal of the hydroxyl group, such as acetyl, benzoyl or the like.

The term “carboxamido”, as used herein, refers to a radical or substituent of the formula —C(═O)—NR″R′″, wherein R′ and R′″ represent hydrogen or alkyl.

The term “sulfonamido”, as used herein, refers to a radical or substituent of the formula —SO₂—NR″R′″ or —NR″—SO₂R′″, wherein R″ and R′″ are as previously defined.

The term “alkanoylamino”, as used herein, refers to a radical or substituent of the formula —NH—C(═O)—R″, wherein R″ is as previously defined.

The term “carbalkoxy”, as used herein, refers to a radical or substituent —C(═O)—OR″, wherein R″ is as previously defined.

The term “bi-heterocyclic group” is used herein to describe a radical comprising two heterocyclic moieties, which may be the same or different, that are chemically linked to one another by a valence bond or a divalent linking moiety such as oxygen or sulfur. See, for instance, entries V9 and V33 in Table V, below. See also, entries V41 and V43.

For the most part, the above-described class of rhodanine derivatives and analogs thereof, as well as their isomers and pharmaceutically acceptable salts exhibit antiviral activity. The compounds of the invention are particularly effective against viruses of the Flaviviridae family and are useful in treating and/or preventing infections and diseases associated with these viruses in living hosts.

The compounds of the invention or precursors thereof and their isomers and pharmaceutically acceptable salts are also useful in treating and preventing viral infections and diseases in living hosts when used in combination with other active agents, including but not limited to interferons, ribavirin, protease inhibitors, immunoglobulins, immunomodulators, hepatoprotectants, anti-inflammatory agents, antibiotics, antivirals, anti-infectious agents, and the like.

Compounds described herein are also useful in preventing or resolving viral infections in cell, tissue or organ cultures and other in vitro applications. For example, inclusion of compounds of the invention as a supplement in cell or tissue culture growth media and cell or tissue culture components will prevent viral infections or contaminations of cultures not previously infected with viruses. Compounds described above may also be used to eliminate viruses from cultures or other biological materials infected or contaminated with viruses (e.g., blood), after a suitable treatment period, under any number of treatment conditions as determined by the skilled artisan.

The compounds of the invention can form useful salts with inorganic and organic acids such as hydrochloric, sulfuric, acetic, lactic, or the like, and with inorganic or organic bases such as sodium or potassium hydroxide, piperidine, morpholine, ammonium hydroxide, or the like. The pharmaceutically acceptable salts of the compounds of formula I are prepared following procedures that are familiar to those skilled in the art.

The antiviral pharmaceutical compositions of the present invention comprise one or more of the compounds of the above-described formulas, as the active ingredient in combination with a pharmaceutically acceptable carrier medium or auxiliary agent and, optionally, one or more supplement active agents, as mentioned above..

The composition may be prepared in various forms for administration, including tablets, caplets, pills or dragees, or can be filled in suitable containers, such as capsules, or, in the case of suspensions, filled into bottles. As used herein, “pharmaceutically acceptable carrier medium” includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Fifteenth Edition, E. W. Martin (Mack Publishing Co., Easton, Pa., 1975) discloses various carriers used in formulating pharmaceutical compositions and known techniques for the preparation thereof. Except insofar as any conventional carrier medium is incompatible with the antiviral compounds of the invention, such as by producing any undesirable biological effect or otherwise interacting in a deleterious manner with any other component(s) of the pharmaceutical composition, its use is contemplated to be within the scope of this invention.

In the pharmaceutical compositions of the invention, the active agent may be present in an amount of at least 0.5% and generally not more than 90% by weight, based on the total weight of the composition, including carrier medium and/or auxiliary agent(s), if any. Preferably, the proportion of active agent varies between 5-50% by weight of the composition.

Pharmaceutical organic or inorganic solid or liquid carrier media suitable for enteral or parenteral administration can be used to make up the composition. Gelatin, lactose, starch, magnesium stearate, talc, vegetable and animal fats and oils, gum, polyalkylene glycol, or other known medicament components may all be suitable as carrier media or excipients.

The compounds of the invention may be administered using any amount and any route of administration effective for attenuating infectivity of the virus. Thus, the expression “amount effective to attenuate infectivity of virus”, as used herein, refers to a nontoxic but sufficient amount of the antiviral agent to provide the desired treatment of viral infection. The exact amount required will vary from subject to subject, depending on the species, age, and general condition of the subject, the severity of the infection, the particular antiviral agent and its mode of administration, and the like.

The antiviral compounds are preferably formulated in dosage unit form for ease of administration and uniformity of dosage. “Dosage unit form” as used herein refers to a physically discrete unit of antiviral agent appropriate for the patient to be treated. Each dosage should contain the quantity of active material calculated to produce the desired therapeutic effect either as such, or in association with the selected pharmaceutical carrier medium and/or the supplemental active agent(s), if any. Typically, the antiviral compounds of the invention will be administered in dosage units containing from about 0.1 mg to about 500 mg of the antiviral agent, with a range of about 1 mg to about 100 mg being preferred.

The compounds of the invention may be administered as such, or in the form of a precursor from which the active agent can be derived, such as a prodrug. A prodrug is a derivative of a compound described herein, the pharmacologic action of which results from the conversion by chemical or metabolic processes in vivo to the active compound. Prodrugs include, without limitation, esters of the compounds described above, having carboxyl or hydroxyl functionalities. Pivaloyloxymethyl esters may be useful for this purpose, as well as esters prepared from simple or functionalized C₁-C₆ alcohols, or from carboxylic acids. Such prodrugs may be prepared according to procedures well known in the field of medicinal chemistry and pharmaceutical formulation science.

The compounds of the invention may be administered orally, rectally, parenterally, such as by intramuscular injection, subcutaneous injection, intravenous infusion or the like, intracisternally, intravaginally, intraperitoneally, locally, such as by powders, ointments, drops or the like, or by inhalation, such as by aerosol or the like, depending on the nature and severity of the infection being treated. Depending on the route of administration, the compounds of the invention may be administered at dosage levels of about 10⁻² to about 120 mg/kg of subject body weight per day and preferably from about 10⁻² to about 30 mg/kg of subject body weight per day, one or more times a day, to obtain the desired therapeutic effect. By way of example, a suitable dose for oral administration would be on the order of 30 mg/kg of body weight per day, whereas a typical intravenous dose would be on the order of 10 mg/kg of body weight per day.

The compounds of the invention will typically be administered from 1 to 4 times a day so as to deliver the above-mentioned daily dosage. However, the exact regimen for administration of the compounds and compositions described herein will necessarily be dependent on the needs of the individual host or patient being treated, the type of treatment administered and the judgment of the attending medical specialist.

In view of the inhibitory effect on viral RNA synthesis produced by the compounds of the invention, it is anticipated that these compounds will be useful not only for therapeutic treatment of virus infection, but for virus infection prophylaxis, as well. The dosages may be essentially the same, whether for treatment or prophylaxis of virus infection.

The following examples are provided to describe the invention in further detail. These examples, which set forth the preferred mode presently contemplated for carrying out the invention, are intended to illustrate and not to limit the invention.

Examples 1-8 illustrate suitable methods of synthesis of representative compounds of this invention. However, the methods of synthesis are not limited to those exemplified below.

### EXAMPLE 1

### Preparation of 5-[5-(2-Chlorophenyl)-furan-2-yl-methylene]-4-oxo-2-thionothiazolidine

a.) 5-Tributyltin-2-Furancarboxaldehyde dimethylacetal—To a solution of 6.03 g (0.0273 moles) of 5-bromo-2-furancarboxaldehyde dimethylacetal (Aldrich) in 75 ml of dry THF at −78° C. under argon was added 12 ml (1. 1 eq) of 2.5 M n-butyl lithium. After 10 minutes, the yellow solution was quenched with 8.88 g (1 eq) of tributyltin chloride, and the reaction slowly allowed to warm to room temperature. After extraction with t-butyl-methyl ether the organic phase was washed with water. After drying of the organic layer over anhydrous sodium sulfate and removal of the solvent 11.3 g (96%) of the product was recovered as an orange oil.

b.) 5-(2-Chlorophenyl)-2-furancarboxaldehyde dimethylacetal—A solution of 2.11 g (4.8 mmoles) of the tributyltin compound obtained from step a.) above and 0.22 ml (1.92 mmoles) of 1-bromo-2-chlorobenzene and 67 mg (5 mole %) of dichlorobis(triphenylphosphine) palladium (II) in 8 ml of redistilled THF was heated to reflux for 12 hours. After cooling, the solution was diluted with 100 ml of diethylether and the mixture filtered through Celite. The organic solution was washed with two portions of water (80 ml each) and dried over anhydrous potassium carbonate. Removal of the solvent gave 264 mg of a brown oil.

c.) 5-(2-Chlorophenyl)-2-furancarboxaldehyde—To a solution of 260 mg (1.03 mmoles) of the dimethylacetal from step b.), above in 5 ml of acetone was added 388 mg (1.54 mmoles) of pyridinium p-toluenesulfonate and the solution was stirred for 12 hours at room temperature. The reaction mixture was diluted with 40 ml of ethyl acetate and the solution washed with two portions of water (30 ml each) and dried over magnesium sulfate. Removal of the solvent provided 123 mg of a yellow solid.

d.) 5-[5-(2-Chlorophenyl)furan-2-yl-methylene]-4-oxo-2-thionothiazolidine—To a solution of 94 mg (0.455 mmoles) of 5-(2-chlorophenyl)-2-furanecarboxaldehyde thus prepared and 64 mg (0.478 mmoles) of rhodanine in 10 ml of ethanol was added 0.1 ml of piperidine, and the solution was heated to reflux. After 20 minutes the solution was cooled and diluted with 80 ml of diethylether and the mixture passed through a fine filter and the redish-orange solid was washed with water and dried under vacuum to provide 116 mg of solid product.

### EXAMPLE 2

### 5-[5-(2-Chloro-5-nitrophenyl)thien-2-yl-methylene]-4-oxo-2-thionothiazolidine

a.) 5-Bromo-2-thiophenecarboxaldehyde dimethylacetal—A solution of 5.9 g (26.2 mmoles) of 5-bromothiophene-2-carboxaldehyde, 3 g (28.3 millimoles) of methylorthoformate and a catalytic amount (10 mg) of pyridinium p-toluene sulfonate in 10 ml of methanol was heated to 40° C. for 48 hours. The solution was concentrated to dryness and purified by flash chromatography on basic alumina by eluting with 4:1 hexane:ethylacetate, providing 5.69 g of product as a clear dark amber oil.

b.) 5-Tributyltin-2-thiophenecarboxaldehyde dimethylacetal—To a solution of 5.6 g (23.6 mmole) of 5-bromo-2-thiophenecarboxaldehyde dimethylacetal in 80 ml of dry tetrahydrofuran at −78° C. under argon was added 10.4 ml (1.1 eq) of 2.5M n-butyllithium. After 15 minutes, the dark orange solution was quenched with 7.69 g (1 eq) of tributyltin chloride. The orange-red solution was allowed to warm to room temperature, and t-butylmethylether (200 ml) was added. The organic phase was washed with two portions of water (100 ml each) and dried over sodium sulfate. Removal of the solvent provided 10.15 g of product as an orange oil.

c.) 5-(2-Chloro-5-nitrophenyl)-2-thiophenecarboxaldehyde dimethylacetal—A suspension of 4.16 g (1.1 eq)) of the tributyltin compound obtained in step b.), above, 2 g (8.4 mmoles) of 1-bromo-2-chloro-5-nitrobenzene and 297 mg (5 mole %) of dichlorobis(triphenylphosphine)palladium (II) in 20 ml of dry THF was heated to reflux under argon for 20 hours. The reaction was concentrated in vacuo and the dark red oil was passed through a basic alumina column. The resulting oil was purified by preparative HPLC through silica gel by eluting with 4:1 hexane:ethyl acetate affording 1.93 g of product as a viscous oil.

d.) 5-(2-Chloro-5-nitrophenyl)-2-thiophenecarboxaldehyde—A solution of 1.93 g of the dimethylacetal from step c.), above, and a catalytic amount (10 mg) of pyridinium p-toluene sulfonate in 100 ml of acetone was stirred in at room temperature under argon for 20 hours. The resulting yellow solution was concentrated to dryness and the residual yellow solid was dissolved in 100 ml of ethyl acetate. The organic phase was washed with two portions of water (100 ml each) and dried over sodium sulfate. Removal of the solvent provided 1.46 g of product as a yellow powder.

e.) 5-[5-(2-Chloro-5-nitrophenyl)thien-2-yl-methylene]-4-oxo-2-thionothiazolidine—A solution of 200mg (0.747 mmoles) of the aldehyde prepared in step d.), above, 104 mg (0.787 mmoles) of 2-thioxo-4-thiazolidinone and 0.02 ml of piperidine in 3 ml of ethanol was heated to reflux for 30 minutes during which time the solution turned dark orange and a solid began to separate. After cooling to room temperature, the mixture was diluted with water and the solid collected by filtration and washed with water. The solid was then heated with ethyl acetate. After cooling to room temperature, the mixture was collected by filtration and dried to give 137 mg of product.

### EXAMPLE 3

### (5-[(5-{2-Chloro-5-trifluoromethylphenyl}furan-2-yl)methylene]-4-oxo-2-thionothiazolidin-3-yl)acetic Acid

To a solution of 191 mg (1 mmole) of rhodanine-3-acetic acid, 274 mg (1 mmole of 5-[2-chloro-5-(trifluoromethyl)phenyl]-2-furaldehyde in 6 ml of ethanol was added 1 drop of piperidine and the solution heated to reflux for 10 minutes. A yellow solid separated and after cooling the mixture, the material was collected by filtration, washed with ethanol and hexane and dried to give 234 mg of the desired product.

### EXAMPLE 4

### 3-(5-[(5-{3,4-Dichlorophenyl}furan-2-yl)methylene]-4-oxo-2-thionothiazolidin-3-yl)propionic Acid

a.) A solution of 5.0 g (28.6 mmoles) of 5-bromo-2-furaldehyde, 4 ml of trimethylorthoformate and 10 mg of pyridinium p-toluenesulfonate in 20 ml of dry methanol was heated to reflux under argon for 18 hours. The solution was concentrated to dryness and the crude yellow oil was passed through basic alumina and the eluent diluted with 4:1 hexane/ethyl acetate to provide 6.03 g of 5-bromo-2-furaldehyde dimethylacetal.

b.) To a solution of 6.03 g (0.0273 moles) of 5-bromo-2-furaldehyde dimethylacetal, prepared as described above, in 75 ml of dry THF at −78° C. under argon was added 12 ml (1.1 eq) of 25 M n-butyl lithium. After 10 minutes, the yellow solution was quenched with 8.88 g (1 eq) of tributyltin chloride, and the reaction slowly allowed to warm to room temperature. After extraction of the solution with water, drying the organic layer over anhydrous sodium sulfate and removal of the solvent, 11.3 g of tri-n-butylstanyl-2-furaldehyde dimethylacetal was obtained as a reddish oil.

c.) To a solution of 8 g (18 mmoles) of the tributyltin compound described above, and 3.5 g (15 mmoles) of 1-bromo-3,4-dichlorobenzene in 25 ml of distilled THF was added 530 mg of palladium (II) chloride bis(triphenylphosphine). The solution was heated to reflux for 12 hours. The reaction mixture was diluted with 100 ml of water and extracted with two portions of ethyl acetate. The organic layer was washed with two portions of water and followed by one portion of saturated sodium chloride and then dried over magnesium sulfate. After filtration, the solution was concentrated and then passed through a silica column and eluted with 9:1 hexane/ethyl acetate to provide 760 mg of 5-(3,4-dichlorophenyl)-2-furaldehyde.

d.) To a solution of 150 mg (0.62 mmoles) of the aldehyde from the previous experiment and 130 mg (0.62 mmoles) of 2-thioxo-4-thiazolidinone-2-proprionic acid in 5 ml of ethanol was added 2 drops of piperidine and the solution heated to reflux for 20 minutes. The solution was diluted with 20 ml of water and 3N HCl was added until the mixture was slightly acidic. The dark orange solid was collected, washed with hexane and dried to yield 198 mg of the desired product.

### EXAMPLE 5

### 6-(5-[(5-{3,4-dichlorophenyl}furan-2-yl)methylene]-4-oxo-2-thionothiazolidin-3-yl)hexanoic Acid

a.) To a mixture of 1 g (4.4 mmole) of trithiocarbonate and 520 mg of 6-aminohexanoic acid was added 5 ml of water and 280 mg of potassium carbonate. The mixture was heated to reflux for 2 hours, then cooled to room temperature and acidified with 3N hydrochloric acid. The mixture was extracted with ethyl acetate and the organic layer washed with water and dried over magnesium sulfate. After filtration, the solvent was removed and the residual solid was suspended in methylene chloride and the suspended solid removed by filtration. The solution was concentrated to dryness and the remaining solid 2-thioxo-4-thiazolidinone-2-hexanoic acid was recrystallized from a mixture of ethyl acetate and hexane.

b.) To 75 mg (0.32 mmole) of the rhodanine hexanoic acid derivative and the furaldehyde, prepared as previously described, in 10 ml of ethanol was added 1 drop of piperidine, and the solution was heated to reflux. After 90 minutes, the mixture was cooled to room temperature and diluted with water and solid separated. The material was collected and dried to give 10 mg of the desired product in solid form.

### EXAMPLE 6

### 3-(5-[(5-{2-Chloro-5-trifluoromethylphenyl}furan-2-yl)methylene]-4-oxo-2-thionothiazolidin-3-yl)benzoic Acid

A solution of 2.48 g (9.8 mmoles) of 3-(4-oxo-2-thioxothiazolidin-3-yl)-benzoic acid, 2.69 g (9.8 mmoles) of 5-([2-chloro-5-trifluoromethylphenyl)-2-furaldehyde, 0.854 ml of piperidine in 150 ml of ethanol was heated to reflux for 4 hours. After cooling to room temperature, the solution was acidified with 1 M hydrochloric acid and the solid which precipitated was collected by filtration, washed with ethanol and dried to give 3.7 g of product.

### EXAMPLE 7

### 4-(5-[5-{2-Chloro-5-trifluoromethylphenyl}furan-2-yl)methylene]-4-oxo-2-thionothiazolidin-3-yl)methylbenzoic Acid

a.) 4-(4-Oxo-2-thioxothiazolidin-3-yl)methylbenzoic Acid

A mixture of 3 g (19.8 mmoles) of 4-(aminomethyl)benzoic acid, 1.05 g (9.98 mmoles) of anhydrous sodium carbonate, and 4.49 g (19.8 mmoles) of bis(carboxymethyl)trithiocarbonate in 50 ml of water was heated to 100° C. for 12 hours. The yellow solid which formed collected by filtration, washed with water and dried to give 4.67 g of product.

b.) 4-(5-[(5-{2-Chloro-5-trifluoromethylphenyl}furan-2-yl)methylene]-4-oxo-2-thionothiazolidin-3-yl)methylbenzoic Acid

A solution of 100 mg (0.374 mmoles) of the methylbenzoic acid derivative prepared in step a.) above, 103 mg (0.374 mmoles) of 5-[2-chloro-5-trifluoromethyl]phenyl)furfural, and 31 mg of piperidine in 10 ml of ethanol was heated to reflux for 20 minutes. The mixture was poured into 10 ml of water and the orange precipitate which formed was collected by filtration, washed with water and dried to give 116 mg of a yellow solid.

Example 8 describes an alternative synthesis for preparing compounds of the present invention.

### EXAMPLE 8

### 4-(5-Phenylmethylene-4-oxo-2-thionothiazolidin-3-yl)benzoic acid

a.) 4-(4-Oxo-2-thionothiazolidin-3-yl)benzoic acid—A mixture of 6.86 g (0.05 moles) of 4-aminobenzoic acid, 11.31 g (0.025 moles) of bis(carboxymethyl)trithiocarbonate and 2.65 g (0.025 moles) of anhydrous sodium carbonate in 50 ml of water was heated to reflux for 12 hours. After cooling to room temperature, the solid which separated was collected and washed with water. After recrystallization, 7.028 g of product was obtained.

b.) 4-(5-Phenylmethylene-4-oxo-2-thionothiazolidin-3-yl)benzoic acid—A solution of 225 mg (0.89 mmoles) of 4-(4-oxo-2-thioxothiazolidin-3-yl)benzoic acid, 0.108 ml (1.07 mmoles) of benzaldehyde, 280 mg of ammonium hydroxide and 309 mg of ammonium chloride in 5 ml of ethanol was heated to reflux for 12 hours. The resulting precipitate was collected by filtration and washed with ethanol affording 72 mg of a yellow solid which melted at 298-301° C.

By appropriate selection of suitable aldehydes or precursors thereof and of specific reactants to provide the desired N-substituent on rhodanine, or an analog thereof, other compounds of the invention may be prepared according to the procedures described in the foregoing examples. Representative examples of further rhodanine derivatives thus prepared are set forth in the tables below.

TABLE I

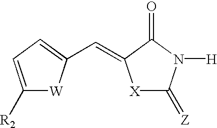

| | R₂ | W | X | Z |
|---|---|---|---|---|
| I-1 | 2-Cl-5-NO₂—Ph* | O | S | S |
| I-2 | 3-CO₂H—Ph | O | S | NH |
| I-3 | 2-Cl-5-NO₂—Ph | O | S | O |
| I-4 | 2-Cl-5-NO₂—Ph | S | S | S |
| I-5 | 2-Cl—Ph | O | S | S |
| I-6 | 3,4-diCl—Ph | O | S | S |
| I-7 | 2-Cl-4-NO₂—Ph | O | S | S |
| I-8 | 4-NO₂—Ph | O | S | NH |
| I-9 | 4-I—Ph | O | S | NH |
| I-10 | 2-Cl-4-NO₂—Ph | O | S | NH |
| I-11 | 4-Cl—Ph | O | S | S |
| I-12 | 2-Cl-5-CF₃—Ph | O | S | S |
| I-13 | 3-Cl—Ph | O | S | S |
| I-14 | 2-Cl-5-CO₂H—Ph | O | S | S |
| I-15 | 3-CO₂H—Ph | O | S | S |
| I-16 | 4-F—Ph | O | S | S |
| I-17 | 4-CH₃O—Ph | O | S | S |
| I-18 | 4-t-butyl-Ph | O | S | S |
| I-19 | 4-O-acetyl-Ph | O | S | S |
| I-20 | Ph | O | S | S |
| I-21 | NO₂ | O | S | S |
| I-22 | H | O | S | S |
| I-23 | 3,4-diCl—Ph | S | S | S |
| I-24 | 3,4-diCl—Ph | O | S | O |
| I-25 | 3,4-diCl—Ph | O | NH | O |
| I-26 | 3,4-diCl—Ph | O | NCH₃ | O |
| I-27 | 2,3-diCl—Ph | S | S | S |
| I-28 | 4-propyl-Ph | S | S | S |

*Ph = phenyl

TABLE II

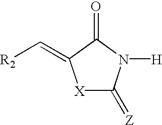

| | R₂ | X | Z |
|---|---|---|---|
| II-1 | bi-Ph(C₆H₅C₆H₄) | S | S |
| II-2 | benzofuran | S | S |

TABLE III

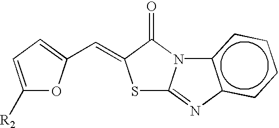

| | R₂ |
|---|---|
| III-1 | 3-Cl-4-Cl—Ph |
| III-2 | 4-CO₂H—Ph |
| III-3 | 4-CH₃O—Ph |

TABLE IV
Rhodaninealkanoic acid derivatives

| | |
|---|---|
| IV-1 | 3-(5-[(5-{2-chloro-5-trifluoromethylphenyl}- |
| | furan-2-yl)methylene]-4-oxo-2-thionothiazolidin- |
| | 3-yl)propionic acid |
| IV-2 | 3-(5-[(5-{3-chlorophenyl}furan-2-yl)methylene]-4-oxo-2- |
| | thionothiazolidin-3-yl)propionic acid |
| IV-3 | (5-[(5-{3,4-dichlorophenyl}furan-2-yl)methylene]-4-oxo-2- |
| | thionothiazolidin-3-yl)acetic acid |
| IV-4 | 4-(5-[(5-{3,4-dichlorophenyl}furan-2-yl)methylene]-4-oxo-2- |
| | thionothiazolidin-3-yl)butyric acid |
| IV-5 | 3-([5-(4-diethylaminophenyl)methylene]-4-oxo-2-thiono |
| | thiazolidin-3-yl)propionic acid |
| IV-6 | 3-([5-(3-phenoxy-4-methoxyphenyl)methylene]-4-oxo-2- |
| | thionothiazolidin-3-yl)propionic acid |
| IV-7 | 3-([5-(3,4-dichlorophenyl)methylene]-4-oxo-2- |
| | thionothiazolidin-3-yl)propionic acid |
| IV-8 | 3-([5-(9-phenanthryl)methylene]-4-oxo-2- |
| | thionothiazolidin-3-yl)propionic acid |
| IV-9 | 3-([5-(2-fluorenyl)methylene]-4-oxo-2- |
| | thionothiazolidin-3-yl)propionic acid |
| IV-10 | (5[(5-{phenyl}furan-2-yl)methylene]-4-oxo- |
| | 2-thionothiazolidin-3-yl)acetic acid |
| IV-11 | 3-(5-[(5-{phenyl}furan-2-yl)methylene]-4-oxo- |
| | 2-thionothiazolidin-3-yl)propionic acid |
| IV-12 | (5-[(5-{3,4-dichlorophenyl}thien-2-yl)methylene]-4-oxo- |
| | 2-thionothiazolidin-3-yl)acetic acid |
| IV-13 | 3-(5-[(5-{phenylethynyl}thien-2-yl)methylene]-4-oxo-2- |
| | thionothiazolidin-3-yl)propionic acid |
| IV-14 | 3-(5-[(5-{thien-2-yl}thien-2-yl)methylene]-4-oxo-2- |
| | thionothiazolidin-3-yl)propionic acid |
| IV-15 | (5-[(5-{3,5-dichlorophenyl}furan-2-yl)methylene]-4-oxo- |
| | 2-thionothiazolidin-3-yl)acetic acid |
| IV-16 | ([5-{(3-para-tert.butyl-phenoxy)-phenyl}methylene]-4-oxo- |
| | 2-thionothiazolidin-3-yl)acetic acid |
| IV-17 | ([5-{3-(4-netrylphenoxy)-phenyl}methylene]-4-oxo-2- |
| | thionothiazolidin-3-yl)acetic acid |
| IV-18 | (5-[((2,5-dimethyl-1-{3-trifluoromethylphenyl})- |
| | 1H-pyrrol-3-yl)methylene]-4-oxo-2-thionothiazolidin- |
| | 3-yl)acetic acid |
| IV-19 | (5-[(5-{3-trifluoromethyl-1-methyl-1H-pyrazol- |
| | 5-yl}thien-2-yl)methylene]-4-oxo-2-thionothiazolidin-3-yl) |
| | 3-acetic acid |
| IV-20 | (5-[((2,5-dimethyl-1{phenyl})1H-pyrrol-3-yl)methylene]-4- |
| | oxo-2-thionothiazolidin-3-yl)acetic acid |
| IV-21 | 5-[(5-{4-chlorophenyl}furan-2-yl)methylene]-4-oxo-2- |
| | thionothiazolidin-3-yl)acetic acid |
| IV-22 | 3-(5-[(5-{3,4-dichlorophenyl}-2-thienyl)methylene]-4-oxo- |
| | 2-thioxothiazolidin-3-yl)propionic acid |
| IV-23 | (5-[(5-{4-carboxylphenyl}furan-2-yl)methylene]-4-oxo- |
| | 2-thionothiazolidin-3-yl)acetic acid |
| IV-24 | (5-[((5-trifluoromethyl-1-methyl-1H-pyrazol- |
| | 3-yl)thien-2-yl)methylene]-4-oxo-2- |
| | thionothiazolidin-3-yl)acetic acid |
| IV-25 | (5-[3-(3-trifluoromethylphenoxy)- |
| | phenylmethylene]-4-oxo-2-thionothiazolidin-3-yl) |
| | acetic acid |
| IV-26 | 3-([5-{4-isopropenyl-cyclohex-1-enyl}methylene]-4-oxo-2- |
| | thionothiazolidin-3-yl)propionic acid |
| IV-27 | 3-([5-(2,4-dichlorophenyl)methylene]-4-oxo-2- |
| | thionothiazolidin-3-yl)propionic acid |
| IV-28 | 3-(5-[(5-(benzofuran-2-yl)furan-2-yl)methylene]-4-oxo-2- |
| | thionothiazolidin-3-yl)propionic acid |
| IV-29 | 3-(5-[(5-{3,5-bistrifluoromethylphenyl}furan-2- |
| | yl)methylene]-4-oxo-2-thionothiazolidin-3-yl)propionic acid |
| IV-30 | 3-(5-[(5-{phenylethnyl}furan-2-yl)methylene]-4-oxo-2- |
| | thionothiazolidin-3-yl)propionic acid |
| IV-31 | 3-(5-[(5-{5-methylpyrid-2-yl}furan-2-yl)methylene]-4-oxo-2- |
| | thionothiazolidin-3-yl)propionic acid |
| IV-32 | 3-(5-[(5-{thiazol-2-yl}furan-2-yl)methylene]-4-oxo-2- |
| | thionothiazolidin-3-yl)propionic acid |
| IV-33 | 5-(4-chlorophenyl)-2-(5-(4-oxo-2-thionothiazolidinyl)-3- |
| | carboxyethyl)-methylenefuran-3-yl-carboxylic acid ethyl ester |
| IV-34 | 5-(4-chlorophenyl)-2-(5-(4-oxo-2-thioxo-4-thiazolidinyl)-3- |
| | carboxymethyl)-methylenefuran-3-yl-carboxylic acid ethyl |
| | ester |
| IV-35 | 3-(5-[(5-(benzothiophen-2-yl)furan-2-yl)methylene]-4-oxo-2- |
| | thionothiazolidinone-3-yl)propionic acid |

TABLE V

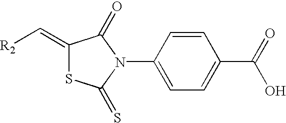

4-(5-(R₂-methylene)-4-oxo-2-thioxo-
thiazolidin-3-yl)benzoic acid

| | R₂ Group |
|---|---|
| V-1 | 5-[2-chloro-5-trifluoromethylphenyl]furan-2-yl |
| V-2 | 5-phenylethynylfuran-2-yl |
| V-3 | 5-[3,4-dichlorophenyl]furan-2-yl |
| V-4 | 5-[4-bromophenyl]furan-2-yl |
| V-5 | 5-[3,5-dichlorophenyl]furan-2-yl |
| V-6 | 5-[2,5-dichlorophenyl]furan-2-yl |
| V-7 | 5-[4-n-butylphenyl]furan-2-yl |
| V-8 | 5-[4-n-propylphenyl]furan-2-yl |
| V-9 | 5-[thien-2-yl]furan-2-yl |
| V-10 | 5-[2-chlorophenyl]furan-2-yl |
| V-11 | 5-[3-carboxyphenyl]furan-2-yl |
| V-12 | 5-[2,3-dichlorophenyl]furan-2-yl |
| V-13 | 5-[3-trifluoromethylphenyl]furan-2-yl |
| V-14 | 5-[2-trifluoromethylphenyl]furan-2-yl |
| V-15 | 5-[2,6-dichlorophenyl]furan-2-yl |
| V-16 | (3-(5-carboxy-2-furanyl)phenyl) |
| V-17 | 4-trans-stilbenyl |
| V-18 | 3-styryl |
| V-19 | 2,4-dichlorophenyl |
| V-20 | 3,4-dichlorophenyl |
| V-21 | 4-bromophenyl |
| V-22 | 4-methoxyphenyl |
| V-23 | 4-carboxyphenyl |
| V-24 | 2-furanyl |
| V-25 | 5-methylfuran-2-yl |
| V-26 | 5-ethylfuran-2-yl |
| V-27 | 4,5-dimethylfuran-2-yl |
| V-28 | 5-[3,5-dimethylphenyl]furan-2-yl |
| V-29 | 5-[3,4-dimethylphenyl]furan-2-yl |
| V-30 | 4-(benzyloxyl)phenyl |
| V-31 | 2-naphthyl |
| V-32 | 5-[3,4-dichlorophenyl]thiophen-2-yl |
| V-33 | 5-[1-methyl-5-trifluoromethyl-1H-pyrazol-3-yl]thiophen-2-yl |
| V-34 | 5-[4-chlorophenyl)furan-2-yl |
| V-35 | 5-[benzofuran-2-yl)furan-2-yl |
| V-36 | 5-[benzothiophen-2-yl]furan-2-yl |
| V-37 | 5-[5-chlorothiophen-2-yl]furan-2-yl |
| V-38 | 5-[3-chloro-5-trifluoromethylpyrid-2-yl]furan-2-yl |
| V-39 | 5-[2,3,5,6-tetrafluoropyrid-4-yl]furan-2-yl |
| V-40 | 5-[6-methoxypyridaz-3-yl]furan-2-yl |
| V-41 | 5-[5-thiazol-2-yl]furan-2-yl |
| V-42 | 5-[2-methyltetrazol-5-yl]furan-2-yl |
| V-43 | 5-(5-trifluoromethyl-[1,2,4]oxadiazol-3-yl)furan-2-yl |

TABLE VI

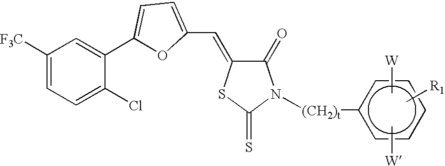

| | R₁ | W | W′ | t |
|---|---|---|---|---|
| VI-1 | 3-COOH | 4-Cl | H | 0 |
| VI-2 | 4-COOH | 3-Cl | H | 0 |
| VI-3 | 4-COO⁻Na⁺ | H | H | 0 |
| VI-4 | 2-COOH | H | H | 0 |
| VI-5 | 3-OH | H | H | 0 |
| VI-6 | 4-COO—CH₂—CH₃ | H | H | 0 |
| VI-7 | H | H | H | 0 |
| VI-8 | 4-SO₂NH₂ | H | H | 0 |
| VI-9 | 4-OH | H | H | 0 |

TABLE VII

| | |
|---|---|
| VII-I | 3-[5-(3-phenylpropenylidenyl)-4-oxo-2- |
| | thionothiazolidin-3-yl]benzoic acid |
| VII-2 | 3-(5-(5-{1-methyl-5-trifluoromethyl-1H-pyrazol-3- |
| | yl}thien-2-yl)methylene-4-oxo-2-thionothiazolidin-3- |
| | yl]benzoic acid |
| VII-3 | 3-[5-(5-{1-methyl-3-trifluoromethyl-1H-pyrazol-5- |
| | yl}thien-2-ylmethylene)-4-oxo-2-thionothiazolidin-3- |
| | yl]benzoic acid |
| VII-4 | 5-(4-chlorophenyl)-2-(3-(4-carboxyphenyl)-4-oxo-2- |
| | thionothiazolidin-5-yl)idenfuran-3-yl carboxylic acid |
| | ethyl ester |

### EXAMPLE 9

### Inhibition of Viral RNA Replication

The discovery of inhibitors of viral polymerases and related proteins generally requires the evaluation of large numbers of chemical compounds or mixtures of chemical compounds. Thus, an assay for the polymerase activity that is capable of high volume screening, in other words, a high-throughput assay, is desirable. There are a variety of assay methodologies well known to the trained artisan that allow the efficient screening of large numbers of samples. See, for example, Cole, J L, Meth Enzymology, 275: 310-328 (1996). Any one of these assays may be suitable in the case of a viral RdRp activity.

One approach for measuring viral RdRp activity in the case of viruses of the Flaviviridae utilizes a purified recombinant NS5 protein in an in vitro RdRp assay. For example, Behrens et al. [EMBO J., 15: 12-22 (1996)] and Lohmann et al. [J Virol, 71:8416-8428 (1997)] describe the baculovirus expression, purification and enzymatic activity of the HCV NS5B RdRp. The bacterial expression, purification and enzymatic activity of the HCV NS5B RdRp protein has been disclosed in PCT/US96/15571 [WO 97/12033] and by Yuan et al. [Bioochem Biophys Res Comm, 232:231-235 (1997)]. In a further example, Collett, PCT/US99/07404, which is commonly owned with the present application, discloses compositions comprising functional HCV NS5B sequences and their use in indentifying compounds useful in the treatment of hepacivirus infections. As with the above examples for the HCV RdRp, bacterially-expressed dengue flavivirus NS5 protein has been purified and shown to exhibit RdRp activity [Tan et al., Virology, 216: 317-325 (1996)], as has the NS5B protein of the pestivirus BVDV purified from recombinant baculovirus-infected cells [Zhong et al., J. Virol., 72: 9365-9369 (1998)].

By way of example, the inhibitory activity of compounds of the invention was demonstrated using NS5 proteins prepared essentially according to Collett, PCT[US99/07404, in in vitro RdRp assays. Purified NS5 proteins were incubated in standard RdRp reaction mixtures. Such reaction mixtures generally consist of buffers, salts, cations, reducing agents and the like, as well as nucleoside triphosphates and an RNA template-primer. Variations in the individual components of such reaction mixtures may be required to accommodate the particular reaction preferences of individual NS5 proteins. Such variations are well known to the trained artisan.

Representative compounds within the scope of the present invention, as shown in Examples 1-8 and the foregoing tables, were evaluated for antiviral activity in this assay. A measure of the inhibitory activity of compounds of the invention may be expressed as IC₅₀ values. IC₅₀ values represent the concentration of the compound at which 50% of the RdRp activity is inhibited. The results of the assay for inhibition of RdRp activity of hepacivirus, pestivirus and flavivirus NS5 proteins for a substantial majority of the compounds tested revealed IC₅₀ values ranging from 0.02 to about 30 μM for each of the three genera.

A number of the compounds tested exhibited IC₅₀ values of ≦1 μM. Such compounds include the following:

A. Rhodanine derivatives of Formula I, above, in which R₁ is hydrogen:

5-[(5-(3,4-dichlorophenyl)furan-2-yl)methylene]-4-oxo-2-thionothiazolidine;

5-[(5-(2-chloro-5-trifluoromethylphenyl)furan-2-yl)methylene]-4-oxo-2-thionothiazolidine;

5-(5-[2-chloro-5-nitrophenyl]furan-2-yl)methylene]-4-oxo-2-thionothiazolidine

5-[(5-(3,4-dichlorophenyl)thien-2-yl)methylene]-4-oxo-2-thionothiazolidine;

5-[(5-(thien-2-yl)thien-2-yl)methylene]-4-oxo-2-thionothiazolidine;

5-[(5-(4-n-propylphenyl)thien-2-yl)methylene]-4-oxo-2-thionothiazolidine;

5-[(5-(4-methylphenyl)thien-2-yl)methylene]-4-oxo-2-thionothiazolidine; and

5-[5-(4,5-dimethylfuran-2-yl)furan-2-yl)methylene]-4-oxo-2-thionothiazolidine.

B. Rhodanine acetic acid derivatives of Formula II above:

(5-[(5-{3,4-dichlorophenyl}furan-2-yl)methylene]-4-oxo-2-thiono-thiazolidin-3-yl)acetic acid;

(5-[(5-{(2-chloro-5-trifluoromethylphenyl}furan-2-yl)methylene]-4-oxo-2-thionothiazolidin-3-yl)acetic acid;

(5-[(5-{3-trifluoromethylphenyl}furan-2-yl)methylene]-4-oxo-2-thionothiazolidin-3-yl)acetic acid;

(5-[(5-{3,5-dichlorophenyl}furan-2-yl)methylene]-4-oxo-2-thionothiazolidin-3-yl)acetic acid;

(5-[(5-{4-chlorophenyl}furan-2-yl)methylene]-4-oxo-2-thionothiazolidin-3-yl)acetic acid;

(5-[(5-{4-chlorophenyl-3-ethoxycarbonyl}furan-2-yl)methylene]-4-oxo-2-thionothiazolidin-3-yl)acetic acid;

(5-[(5-{3,4-dichlorophenyl}thiophen-2-yl)methylene]-4-oxo-2-thionothiazolidin-3-yl)acetic acid; and

(5-[(5-{3-t-butylphenoxyphenyl}furan-2-yl)methylene]-4-oxo-2-thionothiazolidin-3-yl)acetic acid.

C. Rhodanine propionic acid derivatives of Formula II, above:

2-(5-[(5-{2-chloro-5-trifluoromethylphenyl}furan-2-yl)methylene]-4-oxo-2-thionothiazolidin-3-yl)propionic acid;

2-(5-[(5-{5-chlorothiophen-2-yl}furan-2-yl)methylene]-4-oxo-2-thionothiazolidin-3-yl)propionic acid;

3-(5-[(5-{benzofuran-2-yl}furan-2-yl)methylene]-4-oxo-2-thionothiazolidin-3-yl)propionic acid;

3-(5-[(5-{benzothiophen-2-yl}furan-2-yl)methylene]-4-oxo-2-thionothiazolidin-3-yl)propionic acid;

3-(5-[(5-{2-chloro-5-trifluoromethylphenyl}furan-2-yl)methylene]-4-oxo-2-thionothiazolidim-3-yl)propionic acid;

3-(5-[(5-{3,5-ditrifluoromethylpheny}furan-2-yl)methylene]-4-oxo-2-thionothiazolidin-3-yl)propionic acid;

3-(5-[(5-{furan-2-yl}furan-2-yl)methylene]-4-oxo-2-thionothiazolidin-3-yl)propionic acid;

3-(5-[(5-{thiophen-2-yl}furan-2-yl)methylene]-4-oxo-2-thioxothiazolidin-3-yl)propionic acid;

3-(5-[(5-{5-chlorothiophen-2-yl}furan-2-yl)methylene]-4-oxo-2-thionothiazolidin-3-yl)propionic acid;

3-(5-[(-{4-bromophenyl}furan-2-yl)methylene]-4-oxo-2-thionothiazolidin-3-yl)propionic acid;

3-(5-[(5-{isoquinolin-2-yl}furan-2-yl)methylene]-4-oxo-2-thionothiazolidin-3-yl)propionic acid;

3-(5-[(5-{2-trifluoromethylphenyl}furan-2-yl)methylene]-4-oxo-2-thionothiazolidin-3-yl)propionic acid;

3-(5-[(5-{3,4-methylenedioxyphenyl}furan-2-yl)methylene]-4-oxo-2-thionothiazolidin-3-yl)propionic acid;

3-(5-[(5-{3,5-dichlorophenyl}furan-2-yl)methylene]-4-oxo-2-thionothiazolidin-3-yl)propionic acid;

3-(5-[(5-{3,4-dichlorophenyl}furan-2-yl)methylene]-4-oxo-2-thionothiazolidin-3-yl)propionic acid;

3-(5-[(5-{3,5-dimethylphenyl}furan-2-yl)methylene]-4-oxo-2-thionothiazolidin-3-yl)propionic acid;

3-(5-[(5-{5-methylthiophen-2-yl}furan-2-yl)methylene]-4-oxo-thionothiazolidin-3-yl)propionic acid;

3-(5-[(5-{5-methyl-2-pyridyl}furan-2-yl)methylene]-4-oxo-2-thionothiazolidin-3-yl)propionic acid;

3-(5-[(6-benzyloxybenzofuran-2-yl)methylene]-4-oxo-2-thionothiazolidin-3-yl)propionic acid;

3-(5-[(5-{phenanthren-9-yl}furan-2-yl)methylene]-4-oxo-2-thionothiazolidin-3-yl)propionic acid;

3-(5-[(5-{thiophen-2-yl}thiophen-2-yl)methylene]-4-oxo-2-thionothiazolidin-3-yl)propionic acid;

3-(5-[(5-{fluorene-2-yl}furan-2-yl)methylene]-4-oxo-2-thionothiazolidin-3-yl)propionic acid;

3-(5-[(5-{phenylethynyl}thiophen-2-yl)methylene]-4-oxo-2-thionothiazolidin-3-yl)propionic acid;

3-(5-[(5-{3-chlorophenyl}furan-2-yl)methylene]-4-oxo-2-thionothiazolidin-3-yl)propionic acid;

3-(5-[(4-{phenylethyny}thiophen-2-yl)methylene]-4-oxo-2-thionothiazolidin-3-yl)propionic acid;

3-(5-[(5-{5-n-propylthiophen-2-yl}furan-2-yl)methylene]-4-oxo-2-thionothiazolidin-3-yl)propionic acid; and

3-(5-[(5-{4-chlorophenyl}furan-2-yl)methylene]-4-oxo-2-thionothiazolidin-3-yl)propionic acid.

D. Rhodanine benzoic acid derivatives of Formula III, above:

4-(5-[(5-{benzofuran-2-yl}furan-2-yl)methylene]-4-oxo-2-thionothiazolidin-3-yl)benzoic acid;

4-(5-[(5-{benzothiophen-2-yl}furan-2-yl)methylene]-4-oxo-2-thionothiazolidin-3-yl)benzoic acid;

4-(5-[(5-{3,5-ditrifluoromethylphenyl}furan-2-yl)methylene]-4-oxo-2-thionothiazolidin-3-yl)benzoic acid;

4-(5-[(5-{2-chloro-5-trifluoromethylphenyl}furan-2-yl)methylene]-4-oxo-2-thionothiazolidin-3-yl)benzoic acid;

4-(5-[(5-{3,4-dimethylphenyl}furan-2-yl)methylene]-4-oxo-2-thionothiazolidin-3-yl)benzoic acid;

4-(5-[(5-{5-chlorothiophen-2-yl}furan-2-yl)methylene]-4-oxo-2-thionothiazolidin-3-yl)benzoic acid;

4-(5-[(5-{2,5-dichlorophenyl}furan-2-yl)methylene]-4-oxo-2-thionothiazolidin-3-yl)benzoic acid;

4-(5-[(5-{2-chlorophenyl}furan-2-yl)methylene]-4-oxo-2-thionothiazolidin-3-yl)benzoic acid;

4-(5-[(5-{2-furanyl}furan-2-yl)methylene]-4-oxo-2-thionothiazolidin-3-yl)benzoic acid;

4-(5-[(5-{5-methylpyridin-2-yl}furan-2-yl)methylene]-4-oxo-2-thionothiazolidin-3-yl)benzoic acid;

4-(5-[(5-{5-methylthiophen-2-yl}furan-2-yl)methylene]-4-oxo-2-thionothiazolidin-3-yl)benzoic acid;

4-(5-[(5-{3,4-difluorophenyl}furan-2-yl)methylene]-4-oxo-2-thionothiazolidin-3-yl)benzoic acid;

4-(5-[(5-{4-methoxyphenyl}furan-2-yl)methylene]-4-oxo-2-thionothiazolidin-3-yl)benzoic acid;

4-(5-[(5-{5-acetothiophen-2-yl}furan-2-yl)methylene]-4-oxo-2-thionothiazolidin-3-yl)benzoic acid;

4-(5-[(5-{3-chloro-5-trifluoromethylpyridin-2-yl}furan-2-yl)methylene]-4-oxo-2-thionothiazolidin-3-yl)benzoic acid;

4-(5-[(5-{3,4-dimethoxyphenyl}furan-2-yl)methylene]-4-oxo-2-thionothiazolidin-3-yl)benzoic acid;

4-(5-[(5-{3,4-methylenedioxyphenyl}furan-2-yl)methylene]-4-oxo-2-thionothiazolidin-3-yl)benzoic acid;

4-(5-[(5-{5-trifluoromethylpyridin-2-yl}furan-2-yl)methylene]-4-oxo-2-thionothiazolidin-3-yl)benzoic acid;

4-(5-[(5-{6-methoxypyridazin-3-yl}furan-2-yl)methylene]-4-oxo-2-thionothiazolidin-3-yl)benzoic acid;

4-(5-[(5-{4,6-dimethylpyridin-2-yl}furan-2-yl)methylene]-4-oxo-2-thionothiazolidin-3-yl)benzoic acid;

4-(5-[(5-{3-bromo-6-methoxyphenyl}furan-2-yl)methylene]-4-oxo-2-thionothiazolidin-3-yl)benzoic acid;

4-(5-[(5-phenylethynylfuran-2-yl)methylene]-4-oxo-2-thionothiazolidin-yl)benzoic acid;

4-(5-[(5-{3,4-dichlorophenyl}furan-2-yl)methylene]-4-oxo-2-thionothiazolidin-3-yl)benzoic acid;

4-(5-[(5-{4-chlorophenyl}furan-2-yl)methylene]-4-oxo-2-thionothiazolidin-3-yl)benzoic acid;

4-(5-[(5-{4-bromophenyl}furan-2-yl)methylene]-4-oxo-2-thionothiazolidin-3-yl)benzoic acid;

4-(5-[(5-{3,5-dichlorophenyl}furan-2-yl)methylene]-4-oxo-2-thionothiazolidin-3-yl)benzoic acid;

4-(5-[(5-{2-chloro-5-trifluoromethylphenyl}furan-2-yl)methylene]-4-oxo-2-thionothiazolidin-3-yl)-6-chlorobenzoic acid;

4-(5-[(5-{3-carboxyphenyl}furan-2-yl)methylene]-4-oxo-2-thionothiazolidin-3-yl)benzoic acid;

4-(5-[(5-{2-trifluoromethylphenyl}furan-2-yl)methylene]-4-oxo-2-thionothiazolidin-3-yl)benzoic acid;

4-(5-[(5-{2,3,5,6-tetrafluoropyridin-4-yl}furan-2-yl)methylene]-4-oxo-2-thionothiazolidin-3-yl)benzoic acid;

4-5-[(5-{3-trifluoromethylphenyl}furan-2-yl)methylene]-4-oxo-2-thionothiazolidin-3-yl)benzoic acid;

4-(5-[(5-{2-thienyl}furan-2-yl)methylene]-4-oxo-2-thionothiazolidin-3-yl)benzoic acid;

4-(5-[(5-{4-n-butylphenyl}furan-2-yl)methylene]-4-oxo-2-thionothiazolidin-3-yl)benzoic acid;

4-(5-[(5-{2-chloro-5-trifluoromethylphenyl}furan-2-yl)methylene]-4-oxo-2-thionothiazolidin-3-yl)methylenebenzoic acid;

4-(5-[(5-{3,5-difluorophenyl}furan-2-yl)methylene]-4-oxo-2-thionothiazolidin-3-yl)benzoic acid;

4-(5-[(5-{3,5-dimethylphenyl}furan-2-yl)methylene]-4-oxo-2-thionothiazolidin-3-yl)benzoic acid;

4-(5-[(5-{4-acetophenyl}furan-2-yl)methylene]-4-oxo-2-thionothiazolidin-3-yl)benzoic acid;

4-(5-[(5-{4-n-propylphenyl}furan-2-yl)methylene]-4-oxo-2-thionothiazolidin-3-yl)benzoic acid;

4-(5-[(5-{2,3-dichlorophenyl}furan-2-yl)methylene]-4-oxo-2-thionothiazolidin-3-yl)benzoic acid;

4-(5-[(5-{indol-2-yl}furan-2-yl)methylene]-4-oxo-2-thionothiazolidin-3-yl)benzoic acid;

4-(5-[(5-{3-methoxy-2-(N,N-diethylaminocarbonylphenyl)furan-2-yl)methylene]-4-oxo-2-thionothiazolidin-3-yl)benzoic acid;

4-(5-[(5-{phenyl}furan-2-yl)methylene]-4-oxo-2-thionothiazolidin-3-yl)benzoic acid;

4-(5-[(5-{5-methylpyridin-2-yl}furan-2-yl)methylene]-4-oxo-2-thionothiazolidin-3-yl)benzoic acid;

4-(5-[(5-{5-chloro-3-methylbenzothiophen-2-yl}furan-2-yl)methylene]-4-oxo-2-thionothiazolidin-3-yl)benzoic acid;

4-(5-[(5-{5-n-propylthiophen-2-yl}furan-2-yl)methylene]-4-oxo-2-thionothiazolidin-3-yl)benzoic acid;

4-(5-[(5-{4,5-dimethylfuran-2-yl}furan-2-yl)methylene]-4-oxo-2-thionothiazolidin-3-yl)benzoic acid;

4-(5-[(5-{5-thiazol-2-yl}furan-2-yl)methylene]-4-oxo-2-thionothiazolidin-3-yl)benzoic acid;

4-(5-[(5-{formyl}furan-2-yl)methylene]-4-oxo-2-thionothiazolidin-3-yl)benzoic acid;

4-(5-[(5-{4-methylpyridin-2-yl}furan-2-yl)methylene]-4-oxo-2-thionothiazolidin-3-yl)benzoic acid;

4-(5-[(5-{2-acetophenyl}furan-2-yl)methylene]-4-oxo-2-thionothiazolidin-3-yl)benzoic acid;

4-(5-[(5-{2-nitrophenyl}furan-2-yl)methylene]-4-oxo-2-thionothiazolidin-3-yl)benzoic acid; and

4-(5-[(5-{4,5-dichloroimidazol-2-yl}furan-2-yl)methylene]-4-oxo-2-thionothiazolidin-3-yl)benzoic acid.

These low concentrations of test compounds required to achieve 50% inhibition of the RdRp activity indicate that the compounds of the invention are effective at inhibiting RNA synthesis by viral RdRp enzymes.

Although the present invention has been described and exemplified in terms of certain preferred embodiments, other embodiments will be apparent to those skilled in the art. The invention is, therefore, not limited to the particular embodiments described and exemplified, but is capable of modification or variation without departing from the spirit of the invention, the full scope of which is delineated by the appended claims.

What is claimed is:

1. A method of treating or preventing infection caused by at least one virus of the Flaviviridae and disease associated with said infection in a living host having or susceptible to said infection, said method comprising administering to said living host a therapeutically or prophylactically effective amount of a compound, or precursor of said compound, having the formula: 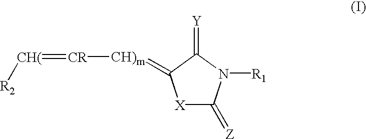
wherein
R represents hydrogen or alkyl; and m is an integer from 0-4;
R₁ represents hydrogen or a radical selected from those consisting of an —R₃COOH radical, wherein R₃ is an unsubstituted or substituted, branched or straight chain, saturated or unsaturated hydrocarbon moiety of 1-10 carbon atoms, an unsubstituted or substituted phenyl (C₆H₅) radical or an unsubstituted or substituted phenylalkyl radical, the R₃ substituents being at least one selected from those consisting of a branched or straight chain, saturated or unsaturated aliphatic group having 1-6 carbon atoms, an unsubstituted or substituted heterocyclic radical or an unsubstituted or substituted phenyl (C₆H₅) radical, said heterocyclic radical being selected from those consisting of furan, thiophene, oxazole, oxadiazole, pyridine, pyrimidine, pyrazole, triazole, pyridazine, 1,3-oxathiolane, thiazole, thiadiazole, imidazole, tetrazole, pyrrole and triazine;
X represents a moiety selected from the group consisting of —S—, —O— or —N(Rₐ)—, Rₐ being hydrogen or alkyl of 1-5 carbon atoms;
R₂ represents a radical selected from those consisting of an unsubstituted or substituted phenylalkyl radical, an unsubstituted or substituted phenylalkenyl radical, an unsubstituted or substituted phenylalkynyl radical, an unsubstituted or substituted biphenylalkyl radical, an unsubstituted or substituted polycyclic radical, an unsubstituted or substituted alycyclic radical having 5-8 carbon atoms or a radical of the formula (R₂ₐ—)ₙ(L—)ₚR_{2b}—, wherein R₂ₐ and R_{2b} may be the same or different and represent an unsubstituted or substituted heterocyclic radical or an unsubstituted or substituted phenyl radical, R₂ₐ also represents an unsubstituted or substituted polycyclic radical and L represents a divalent linking moiety selected from the group consisting of a valence bond, —(CH₂)_{q}—, —HC═CH—, —C═C—, —C(═O)—, —O—, —S—, —S(═O)—, —S(═O)₂—, NR_{2c}, R_{2c} being hydrogen or alkyl, n and p are each 0 or 1, and q is an integer from 1 to 3;
said heterocyclic radicals being selected from the group consisting of furan, thiophene, oxazole, oxadiazole, isoxazole, pyridine, pyrimidine, pyrazole, triazole, pyridazine, 1,3-oxathiolane, thiazole, isothiazole, thiadiazole, imidazole, pyrrole, tetrazole and triazine;
said polycyclic radicals being selected from the group consisting of benzofuran, isobenzofuran, benzothiophene, isobenzothiophene, benzoxazole, indole, 2-isoindole, benzopyrazole, quinoline, isoquinoline, 1,2-benzodiazine, 1,3-benzodiazine, 1,2,3-benzotriazole, benzothiazole, benzimidazole, 1,2,3-benzotriazine, 1,2,4-benzotriazine, naphthalene, anthracene and fluorene;
the heterocyclic radical substituents, the polycyclic radical substituents and the alicyclic radical substituents being at least one selected from the group consisting of a straight or branched chain, saturated or unsaturated aliphatic group having 1-6 carbon atoms, halogen, perhaloalkyl, monoalkyl, dihaloalkyl, alkoxy, acyl, acyloxy, acyloxyalkyl, phenylalkoxy, hydroxy, hydroxyalkyl, thio, alkylthio, nitro, carboxy, carbalkoxy;
the phenyl radical substituents, the phenylalkyl radical substituents, the phenylalkenyl radical substituents, the phenylalkynyl radical substituents and the biphenylalkyl radical substituents being at least one selected from the group consisting of a straight or branched chain, saturated or unsaturated aliphatic group having 1-6 carbon atoms, halogen, nitro, carboxy, hydroxy, hydroxyalkyl, perhaloalkyl, monohaloalkyl, dihaloalkyl, alkoxy, phenylalkoxy, acyl, acyloxy, acyloxyalkyl, cyano, carbalkoxy, thio, alkylthio, alkylsulfinyl, alkylsulfonyl, amino, alkylamino, dialkylamino, aminoalkyl, alkylaminoalkyl, dialkylaminoalkyl, sulfonamido, carboxamido, alkanoylamino;
Y represents O or S;
Z represents O, S or N(R_{b}), R_{b} being hydrogen or alkyl; or R₁ and R_{b} may be joined to form an imidazole or a benzimidazole moiety; and the isomers and pharmaceutically acceptable salts of said compound.

2. A method as claimed in claim 1, wherein said compound or a precursor of said compound is administered to a living host in unit dosage form containing from about 10⁻³ to about 120 mg of said compound per kilogram of body weight per day, said unit dosage optionally including a pharmaceutically acceptable carrier medium.

3. A method as claimed in claim 1, wherein a precursor of said compound is administered in the form of a prodrug.

4. A method as claimed in claim 1, wherein said compound or precursor of said compound is administered together, either simultaneously or sequentially, with at least one other therapeutic agent.

5. A method as claimed in claim 4, wherein said other therapeutic agent is selected from the group consisting of interferons, ribavirin, protease inhibitors, immunoglobulins, immunomodulators, hepatoprotectants, anti-inflammatory agents, antibiotics, antivirals and anti-infectious agents.

6. A method as claimed in claim 1, wherein said compound or a precursor of said compound is administered orally.

7. A method as claimed in claim 1, wherein said compound or a precursor of said compound is administered rectally.

8. A method as claimed in claim 1, wherein said compound or a precursor of said compound is administered parenterally.

9. A method as claimed in claim 1, wherein said compound or a precursor of said compound is administered intracisternally.

10. A method as claimed in claim 1, wherein said compound or a precursor of said compound is administered intravaginally.

11. A method as claimed in claim 1, wherein said compound or a precursor of said compound is administered intraperitoneally.

12. A method as claimed in claim 1, wherein said compound or a precursor of said compound is administered locally.

13. A method as claimed in claim 1, wherein said compound or a precursor of said compound is administered by inhalation.

14. A method as claimed in claim 1, wherein said viruses of the Flaviviridae family are selected from the group consisting of viruses of the hepacivirus genus, viruses of the pestivirus genus, viruses of the flavivirus genus and viruses unassigned to particular genera within the Flaviviridae family.

15. A method as claimed in claim 14, wherein said compound or a precursor of said compound is administered to living hosts in unit dosage form containing about 10⁻³ to about 120 mg of said compound per kilogram of body weight per day.

16. A method as claimed in claim 14, wherein a precursor of said compound is administered in the form of a prodrug.

17. A method as claimed in claim 1, wherein said compound has the formula: 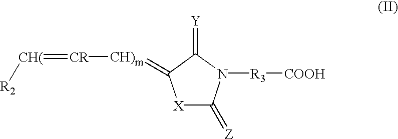
wherein
R represents hydrogen or alkyl; and m is an integer from 0-4;
R₃ represents an unsubstituted or substituted, branched or straight chain, saturated or unsaturated hydrocarbon moiety having 1-10 carbon atoms in the main chain, said hydrocarbon moiety substituents being at least one selected from those consisting of a branched or straight chain, saturated or unsaturated aliphatic group, having 1-6 carbon atoms, an unsubstituted or substituted mono-heterocyclic radical or an unsubstituted or substituted phenyl (C₆H₅) radical, said mono-heterocyclic radical being selected from those consisting of furan, thiophene, oxazole, oxadiazole, isoxazole, pyridine, pyrimidine, pyrazole, triazole, pyridazine, 1,3-oxathiolane, thiazole, thiadiazole, imidazole, tetrazole, pyrrole and triazine;
X represents a moiety selected from the group consisting of —S—, —O—, or —(Rₐ)—, Rₐ being hydrogen or alkyl;
R₂ represents a radical selected from those consisting of an unsubstituted or substituted phenylalkyl radical, an unsubstituted or substituted phenylalkenyl radical, an unsubstituted or substituted phenylalkynyl radical, an unsubstituted or substituted biphenylalkyl radical, an unsubstituted or substituted polycyclic radical, an unsubstituted or substituted alycyclic radical having 5-8 carbon atoms or a radical of the formula (R₂ₐ—)ₙ(L—)ₚR_{2b}—, wherein R₂ₐ and R_{2b} may be the same or different and represent an unsubstituted or substituted heterocyclic radical or an unsubstituted or substituted phenyl radical, R₂ₐ also represents an unsubstituted or substituted polycyclic radical and L represents a divalent linking moiety selected from the group consisting of a valence bond, —(CH₂)_{q}—, —HC═CH—, —C═C—, —C(═O)—, —O—, —S—, —S(═O)—, —S(═O)₂—, NR_{2c}, R_{2c} being hydrogen or alkyl, n and p are each 0 or 1, and q is an integer from 1 to 3;
said heterocyclic radicals being selected from the group consisting of furan, thiophene, oxazole, oxadiazole, isoxazole, pyridine, pyrimidine, pyrazole, triazole, pyridazine, 1,3-oxathiolane, thiazole, isothiazole, thiadiazole, imidazole, pyrrole, tetrazole and triazine;
said polycyclic radicals being selected from the group consisting of benzofuran, isobenzofuran, benzothiophene, isobenzothiophene, benzoxazole, indole, 2-isoindole, benzopyrazole, quinoline, isoquinoline, 1,2-benzodiazine, 1,3-benzodiazine, 1,2,3-benzotriazole, benzothiazole, benzimidazole, 1,2,3-benzotriazine, 1,2,4-benzotriazine, naphthalene, anthracene and fluorene;
the heterocyclic radical substituents, the polycyclic radical substituents and the alicyclic radical substituents being at least one selected from the group consisting of a straight or branched chain, saturated or unsaturated aliphatic group having 1-6 carbon atoms, halogen, perhaloalkyl, monoalkyl, dihaloalkyl, alkoxy, acyl, acyloxy, acyloxyalkyl, phenylalkoxy, hydroxy, hydroxyalkyl, thio, alkylthio, nitro, carboxy, carbalkoxy;
the phenyl radical substituents, the phenylalkyl radical substituents, the phenylalkenyl radical substituents, the phenylalkynyl radical substituents and the biphenylalkyl radical substituents being at least one selected from the group consisting of a straight or branched chain, saturated or unsaturated aliphatic group having 1-6 carbon atoms, halogen, nitro, carboxy, hydroxy, hydroxyalkyl, perhaloalkyl, monohaloalkyl, dihaloalkyl, alkoxy, phenylalkoxy, acyl, acyloxy, acyloxyalkyl, cyano, carbalkoxy, thio, alkylthio, alkylsulfinyl, alkylsulfonyl, amino, alkylamino, dialkylamino, aminoalkyl, alkylaminoalkyl, dialkylaminoalkyl, sulfonamido, carboxamido, alkanoylamino;
Y represents O or S;
Z represents O, S or N(R_{b}), R_{b} being hydrogen or alkyl;
or R₃ and R_{b} may be jointed to form an imidazole or benzimidazole moiety; and the isomers and pharmaceutically acceptable salts of said compound.

18. A method as claimed in claim 17, wherein said compound is selected from the group consisting of 3-(5-[(5-{benzofuran-2-yl}furan-2-yl)methylene]-4-oxo-2-thionothiazolidin-3-yl-propionic acid; 3-(5-[(5-{benzothiophen-2-yl}-furan-2-yl)methylene]-4-oxo-2-thionothiazolidin-3-yl-propionic acid; 3-(5-[(5-(2-chloro-5-trifluoromethylphenyl)-2-furanyl)methylene]-4-oxo-2-thionothiazolidin-3-yl-propionic acid; 3-(5-[(5-(5-chlorothiophen-2-yl)furan-2-yl)-methylene-4-oxo-2-thionothiazolidin-3-yl)propionic acid.

19. A method as claimed in claim 1, wherein said compound has the formula: 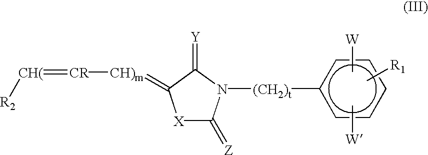
wherein
R represents hydrogen or alkyl; and m is an integer from 0-4;
R₁ represents hydrogen or a substituent selected from the group consisting of —OH, —COOR₄, —CONR₅R₆, —SO₂NR₇R₈, R₄, R₅, R₆, R₇ and R₈ being independently selected from the group of hydrogen or alkyl, or R₁ represents a mono-heterocylic radical selected from the group of furan, thiophene, pyridine, pyrimidine, pyridazine, 1,3-oxathiolane, tetrazole, oxadiazole, oxazole, triazole, imidazoline, imidazole, thiazole, thiadiazole, pyrrole, piperidine, morpholine, triazine and pyrazole;
W and W′ may be the same or different and represent hydrogen or a substituent selected from the group consisting of a straight or branched chain, saturated or unsaturated aliphatic group having 1-6 carbon atoms, halogen, nitro, hydroxy, perfluoroalkyl, difluoromethyl, alkoxy, phenoxy, phenylalkoxy, acyl, acyloxy, acyloxyalkyl, cyano, carbalkoxy, thio, alkylthio, alkylsulfinyl, phenylsulfonyl, alkylsulfonyl, amino, alkylamino, dialkylamino, sulfonamido, carboxamido and alkanoylamino.
t is an integer from 0 to 8;
X represents a moiety selected from the group consisting of —S—, —O— or —N(Rₐ)—, Rₐ being hydrogen or alkyl;
R₂ represents a radical selected from those consisting of an unsubstituted or substituted phenylalkyl radical, an unsubstituted or substituted phenylalkenyl radical, an unsubstituted or substituted phenylalkynyl radical, an unsubstituted or substituted biphenylalkyl radical, an unsubstituted or substituted polycyclic radical, an unsubstituted or substituted alycyclic radical having 5-8 carbon atoms or a radical of the formula (R₂ₐ—)ₙ(L—)ₚR_{2b}—, wherein R₂ₐ and R_{2b} may be the same or different and represent an unsubstituted or substituted heterocyclic radical or an unsubstituted or substituted phenyl radical, R₂ₐ also represents an unsubstituted or substituted polycyclic radical and L represents a divalent linking moiety selected from the group consisting of a valence bond, —(CH₂)_{q}—, —HC═CH—, —C═C—, —C(═O)—, —O—, —S—, —S(═O)—, —S(═O)₂—, NR_{2c}, R_{2c} being hydrogen or alkyl, n and p are each 0 or 1, and q is an integer from 1 to 3;
said heterocyclic radicals being selected from the group consisting of furan, thiophene, oxazole, oxadiazole, isoxazole, pyridine, pyrimidine, pyrazole, triazole, pyridazine, 1,3-oxathiolane, thiazole, isothiazole, thiadiazole, imidazole, pyrrole, tetrazole and triazine;
said polycyclic radicals being selected from the group consisting of benzofuran, isobenzofuran, benzothiophene, isobenzothiophene, benzoxazole, indole, 2-isoindole, benzopyrazole, quinoline, isoquinoline, 1,2-benzodiazine, 1,3-benzodiazine, 1,2,3-benzotriazole, benzothiazole, benzimidazole, 1,2,3-benzotriazine, 1,2,4-benzotriazine, naphthalene, anthracene and fluorene;
the heterocyclic radical substituents, said polycyclic radical substituents and said alicyclic radical substituents being at least one selected from the group consisting of a straight or branched chain, saturated or unsaturated aliphatic group having 1-6 carbon atoms, halogen, perhaloalkyl, monoalkyl, dihaloalkyl, alkoxy, acyl, acyloxy, acyloxyalkyl, phenylalkoxy, hydroxy, hydroxyalkyl, thio, alkylthio, nitro, carboxy, carbalkoxy;
the phenyl radical substituents, the phenylalkyl radical substituents, the phenylalkenyl radical substituents, the phenylalkynyl radical substituents and the biphenylalkyl radical substituents being at least one selected from the group consisting of a straight or branched chain, saturated or unsaturated aliphatic group having 1-6 carbon atoms, halogen, nitro, carboxy, hydroxy, hydroxalkyl, perhaloalkyl, monohaloalkyl, dihaloalkyl, alkoxy, phenylalkoxy, acyl, acyloxy, acyloxyalkyl, cyano, carbalkoxy, thio, alkylthio, alkylsulfinyl, alkylsulfonyl, amino, alkylamino, dialkylamino, aminoalkyl, alkylaminoalkyl, dialkylaminoalkyl, sulfonamido, carboxamido, alkanoylamino;
Y represents O or S;
Z represents O, S or N(R_{b}), R_{b} being hydrogen or alkyl;
or R₁ and R_{b} may be joined to form an imidazole or benzimidazole moiety; and the isomers and pharmaceutically acceptable salts of said compound.

20. A method as claimed in claim 19, wherein said compound is selected from the group consisting of 4-(5-(5-[2-chloro-5-trifluoromethylphenyl]furan-2-yl-methylene)4-oxo-2-thionothiazolidin-3-yl)benzoic acid; 4-(5-(5-[3,4-dichlorophenyl]furan-2-yl-methylene)-4-oxo-2-thionothiazolidin-3-yl)benzoic acid; 4-(5-(5-[benzothiophen-2-yl]furan-2-yl-methylene)-4-oxo-2-thionothiazolidin-3-yl)benzoic acid; 4-(5-(5-[benzofuran-2-yl]furan-2-yl-methylene)-4-oxo-2-thionothiazolidin-3-yl)benzoic acid; 4-(5-{5-[3-bromo-6-methoxyphenyl]furan-2-yl-methylene}-4-oxo-2-thionothiazolidin-3-yl)benzoic acid; 4-(5-(4-[5-chlorothiophen-2-yl]thiazol-2-yl-methylene)-4-oxo-2-thionothiazolidin-3-yl)benzoic acid; 4-(5-(5-[3,5-ditrifluoromethylphenyl]furan-2-yl-methylene)-4-oxo-2-thionothiazolidin-3-yl)benzoic acid.

21. A method of treating infection caused by at least one virus of the hepacivirus genus of Flaviviridae and disease associated with said infection in a patient in need of said treatment, said method comprising administering to said patient a therapeutically effective amount of a compound, or a precursor of said compound, having the formula: 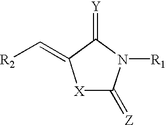
wherein
R₁ represents hydrogen or a radical selected from those consisting of R₃COOH, wherein R₃ is a branched or straight chain aliphatic moiety of 1-10 carbon atoms, or an unsubstituted, or substituted phenyl (C₆H₅) group;
X represents a moiety selected from the group consisting of —S—, —O— or —N(Rₐ)—, Rₐ being hydrogen or alkyl of 1-5 carbon atoms;
R₂ represents a radical selected from those consisting of an unsubstituted or substituted hetero-cyclic group, an unsubstituted or substituted bicyclic ring moiety, an unsubstituted or substituted phenyl group, an unsubstituted or substituted biphenyl (C₆H₅—C₆H₄) group or an unsubstituted or substituted cinnamenyl (C₆H₅CH═CH—) group, said heterocyclic group being selected from those consisting of furan, thiophene, oxadiazole, pyridine, pyrimidine, pyrazole, triazole, pyridazine, 1,3-oxathiolane, thiazole, thiadiazole, imidazole, pyrrole and triazine, said bicyclic ring moiety being selected from those consisting of benzofuran, isobenzofuran, benzothiophene, isobenzothiophene, benzoxazole, benzopyrrole, indolenine, 2-isobenzazole, benzpyrazole, quinoline, isoquinoline, 1,2-benzodiazine, 1,3-benzodiazine, 1,2,3-benzotriazole, benzothiazole, benzimidazole, 1,2,3-benzotriazine and 1,2,4-benzotriazine, the heterocyclic group and bicyclic ring moiety substituents being at least one selected from those consisting of alkyl of 1-5 carbon atoms, halogen, alkoxy, hydroxy, nitro or an unsubstituted or substituted phenyl group;
the phenyl group substituents, the biphenyl group substituents and the cinnamenyl group substituents being at least one selected from those consisting of halogen, nitro, carboxy, hydroxy, alkyl of 1-5 carbon atoms, trifluoromethyl, alkoxy, acyloxy, cyano, carbalkoxy, alkylthio, alkylsulfinyl, alkylsulfonyl, amino, alkylamino, dialkylamino, sulfonamido or carboxamido;
Y represents O or S;
Z represents O, S or N(R_{b}), R_{b} being hydrogen or alkyl of 1-5 carbon atoms;
or R₁ and R_{b} may be joined to form a benzimidazole moiety; and the isomers and pharmaceutically acceptable salts of said compound.

22. A pharmaceutical composition for treating or preventing viral infections, said composition comprising an anti-viral agent in an amount effective to attenuate viral infectivity, and a pharmaceutically acceptable carrier medium, said anti-viral agent comprising a compound of the formula: 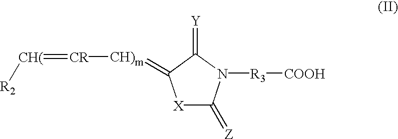
wherein
R represents hydrogen or alkyl; and m is an integer from 0-4;
R₃ represents an unsubstituted or substituted, branched or straight chain, saturated or unsaturated hydrocarbon moiety having 1-10 carbon atoms in the main chain, said hydrocarbon moiety substituents being at least one selected from those consisting of a branched or straight chain, saturated or unsaturated aliphatic group, having 1-6 carbon atoms, an unsubstituted or substituted heterocyclic radical or an unsubstituted or substituted phenyl (C₆H₅) radical, said heterocyclic radical being selected from those consisting of furan, thiophene, oxazole, oxadiazole, pyridine, pyrimidine, pyrazole, triazole, pyridazine, 1,3-oxathiolane, thiazole, thiadiazole, imidazole, tetrazole, pyrrole and triazine;
X represents a moiety selected from the group consisting of —S—, —O—, or —N(Rₐ)—, Rₐ being hydrogen or alkyl;
R₂ represents a radical selected from those consisting of an unsubstituted or substituted phenylalkyl radical, an unsubstituted or substituted phenylalkenyl radical, an unsubstituted or substituted phenylalkynyl radical, an unsubstituted or substituted biphenylalkyl radical, an unsubstituted or substituted polycyclic radical, an unsubstituted or substituted alycyclic radical having 5-8 carbon atoms or a radical of the formula (R₂ₐ—)ₙ(L—)ₚR_{2b}—, wherein R₂ₐ and R_{2b} may be the same or different and represent an unsubstituted or substituted heterocyclic radical or an unsubstituted or substituted phenyl radical, R₂ₐ also represents an unsubstituted or substituted polycyclic radical and L represents a divalent linking moiety selected from the group consisting of a valence bond, —(CH₂)_{q}—, —HC═CH—, —C═C—, —C(═O)—, —O—, —S—, —S(═O)—, —S(═O)₂—, NR_{2c}, R_{2c} being hydrogen or alkyl, n and p are each 0 or 1, and q is an integer from 1 to 3;
said heterocyclic radicals being selected from the group consisting of furan, thiophene, oxazole, oxadiazole, isoxazole, pyridine, pyrimidine, pyrazole, triazole, pyridazine, 1,3-oxathiolane, thiazole, isothiazole, thiadiazole, imidazole, pyrrole, tetrazole and triazine;
said polycyclic radicals being selected from the group consisting of benzofuran, isobenzofuran, benzothiophene, isobenzothiophene, benzoxazole, indole, 2-isoindole, benzopyrazole, quinoline, isoquinoline, 1,2-benzodiazine, 1,3-benzodiazine, 1,2,3-benzotriazole, benzothiazole, benzimidazole, 1,2,3-benzotriazine, 1,2,4-benzotriazine, naphthalene, anthracene and fluorene;
the heterocyclic radical substituents, said polycyclic radical substituents and said alicyclic radical substituents being at least one selected from the group consisting of a straight or branched chain, saturated or unsaturated aliphatic group having 1-6 carbon atoms, halogen, perhaloalkyl, monoalkyl, dihaloalkyl, alkoxy, acyl, acyloxy, acyloxyalkyl, phenylalkoxy, hydroxy, hydroxyalkyl, thio, alkylthio, nitro, carboxy, carbalkoxy;
the phenyl radical substituents, the phenylalkyl radical substituents, the phenylalkenyl radical substituents, the phenylalkynyl radical substituents and the biphenylalkyl radical substituents being at least one selected from the group consisting of a straight or branched chain, saturated or unsaturated aliphatic group having 1-6 carbon atoms, halogen, nitro, carboxy, hydroxy, hydroxyalkyl, perhaloalkyl, monohaloalkyl, dihaloalkyl, alkoxy, phenylalkoxy, acyl, acyloxy, acyloxyalkyl, cyano, carbalkoxy, thio, alkylthio, alkylsulfinyl, alkylsulfonyl, amino, alkylamino, dialkylamino, aminoalkyl, alkylaminoalkyl, dialkylaminoalkyl, sulfonamido, carboxamido, alkanoylamino;
Y represents O or S;
Z represents O, S or N(R_{b}), R_{b} being hydrogen or alkyl;
or R₃ and R_{b} may be jointed to form an imidazole or benzimidazole moiety; and the isomers and pharmaceutically acceptable salts of said compound.

23. A composition as claimed in claim 22, wherein said R₂ radical is of the formula: (R₂ₐ—)ₙ(L—)ₚR_{2b}—, p is 0; and m is 0.

24. A composition as claimed in claim 22 comprising a compound of formula II, wherein R₃ is a straight chain alkylene of 1-5 carbon atoms, Y is oxygen, X and Z are sulfur and R₂ is an unsubstituted or substituted mono-heterocyclic radical selected from those consisting of furan, thiophene and oxazole, or an unsubstituted or substituted bi-heterocyclic radical selected from those consisting of bi-thienyl and 1H-pyrazolylthienyl, the heterocyclic radical substituents being at least one selected from those consisting of halogen, trifluoromethyl or an unsubstituted or substituted phenyl radical, and said phenyl radical substituents being at least one selected from those consisting of halogen, nitro, carboxy, hydroxy,methyl, ethyl, trifluoromethyl, difluoromethyl, alkoxy, phenoxy, acyloxy, cyano, carbalkoxy, thio, alkylthio, alkylsulfinyl, alkylsulfonyl, amino, alkylamino, dialkylamino, sulfonamido, carboxamido, alkanoylamino, 1-pyrrolidinyl, 1-piperidinyl or 4-morpholinyl.

25. A composition as claimed in claim 22 comprising a compound of formula II, wherein R₃ is a straight chain alkylene of 1-5 carbon atoms, Y is oxygen, X and Z are sulfur and R₂ is an unsubstituted or substituted phenyl radical, the phenyl radical substituents being at least one selected from those consisting of halogen, nitro, carboxy, hydroxy,methyl, ethyl, trifluoromethyl, difluoromethyl, alkoxy, phenoxy, acyloxy, cyano, carbalkoxy, thio, alkylthio, alkylsulfinyl, alkylsulfonyl, amino, alkylamino, dialkylamino, sulfonamido, carboxamido, alkanoylamino, 1-pyrrolidinyl, 1-piperidinyl or 4-morpholinyl.

26. A composition as claimed in claim 22 comprising a compound of formula II, wherein R₃ is a straight chain alkylene of 1-5 carbon atoms, Y is oxygen, X and Z are sulfur and R₂ is an unsubstituted or substituted polycyclic radical selected from those consisting of 9-phenanthryl and 2-fluorenyl, said polycyclic radical substituents being at least one selected from those consisting of methyl, ethyl, halogen, alkoxy, hydroxy, thio, nitro or an unsubstituted or substituted phenyl radical, said the phenyl radical substituents being at least one selected from those consisting of halogen, nitro, carboxy, hydroxy,methyl, ethyl, trifluoromethyl, difluoromethyl, alkoxy, phenoxy, acyloxy, cyano, carbalkoxy, thioalkylthio, alkylsulfinyl, alkylsulfonyl, amino, alkylamino, dialkylamino, sulfonamido, carboxamido, alkanoylamino, 1-pyrolidyl, 1-piperidinyl or 4-morpholinyl.

27. A composition as claimed in claim 22 comprising a compound of formula II, selected from the group consisting of 3-(5-[(5-{benzofuran-2-yl}furan-2-yl)methylene]-4-oxo-2-thionothiazolidin-3-yl-propionic acid; 3-(5-[(5-{benzothiophen-2-yl}-furan-2-yl)methylene]-4-oxo-2-thionothiazolidin-3-yl-propionic acid; 3-(5-[(5-(2-chloro-5-trifluoromethylphenyl)-2-furanyl)methylene]-4-oxo-2-thionothiazolidin-3-yl-propionic acid; 3-(5-[(5-(5-chlorothiophen-2-yl)furan-2-yl)-methylene-4-oxo-2-thionothiazolidin-3-yl)propionic acid.

28. A composition as claimed in claim 22 further including at least one therapeutic agent selected from the group consisting of interferons, ribavirin, protease inhibitors, immunoglobulins, immunomodulators, hepatoprotectants, anti-inflammatory agents, antibiotics, antivirals and anti-infectious agents.

29. A pharmaceutical composition for treating or preventing viral infections, said composition comprising an anti-viral agent in an amount effective to attenuate viral infectivity, and a pharmaceutically acceptable carrier medium, said anti-viral agent comprising a compound of the formula: 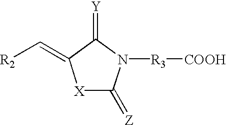
wherein
R₃ represents an unsubstituted or substituted, branched or straight chain, saturated or unsaturated aliphatic moiety having 1-10 carbon atoms in the main chain, said aliphatic moiety substituents being selected from those consisting of at least one branched or straight chain, saturated or unsaturated aliphatic group, having 1-6 carbon atoms, unsubstituted or substituted mono-heterocyclic group or unsubstituted or substituted phenyl (C₆H₅) group, said heterocyclic group being selected from those consisting of furan, thiophene, oxazole, oxadiazole, pyridine, pyrimidine, pyrazole, triazole, pyridazine, 1,3-oxathiolane, thiazole, thiadiazole, imidazole, tetrazole, pyrrole and triazine;
X represents a moiety selected from the group consisting of —S—, —O— or —N(Rₐ)—, Rₐ being hydrogen or alkyl;
R₂ represents a radical selected from those consisting of an unsubstituted or substituted mono- or bi-heterocyclic group, an unsubstituted or substituted polycyclic ring moiety, an unsubstituted or substituted alicyclic group having 5-8 carbon atoms, an unsubstituted or substituted phenyl group, an unsubstituted or substituted biphenyl (C₆H₅—C₆H₄—) group, an unsubstituted or substituted phenyl ether group (C₆H₅—O—C₆H₄—) or an unsubstituted or substituted cinnamyl (C₆H₅CH═CH—) group, said mono-heterocyclic group being selected from those consisting of furan, thiophene, oxazole, oxadiazole, pyridine, pyrimidine, pyrazole, triazole, pyridazine, 1,3-oxathiolane, thiazole, thiadiazole, imidazole, tetrazole, pyrrole and triazine, said bi-heterocyclic group comprising two heterocyclic groups which are selected from said mono-heterocyclic group members, and which may be the same or different, said polycyclic ring moiety being selected from those consisting of benzofuran, isobenzofuran, benzothiophene, isobenzothiophene, benzoxazole, benzopyrrole, indolenine, 2-isobenzazole, benzpyrazole, quinoline, isoquinoline, 1,2-benzodiazine, 1,3-benzodiazine, 1,2,3-benzotriazole, benzothiazole, benzimidazole, 1,2,3-benzotriazine and 1,2,4-benzotriazine, naphthalene, anthracene and fluorene;
the mono- or bi-heterocyclic group substituents, the alicyclic group substituents and the polycyclic ring moiety substituents being at least one selected from those consisting of a straight or branched chain, saturated or unsaturated aliphatic group having 1-6 carbon atoms, halogen, trifluoromethyl, alkoxy, hydroxy, thio, nitro, an unsubstituted or substituted phenyl group, an unsubstituted or substituted phenylalkenyl group or an unsubstituted or substituted phenylalkynyl group;
the phenyl group substituents, the biphenyl group substituents, the phenyl ether group substituents, the phenylalkenyl group substituents, the phenylalkynyl group substituents and the cinnamenyl group substituents being at least one selected from those consisting of a straight or branched chain, saturated or unsaturated aliphatic group having 1-6 carbon atoms, halogen, nitro, carboxy, hydroxy, trifluoromethyl, difluoromethyl, alkoxy, phenoxy, acyloxy, cyano, carbalkoxy, thio, alkylthio, alkylsulfinyl, alkylsulfonyl, amino, alkylamino, dialkylamino, sulfonamido, carboxamido, alkanoylamino, 1-pyrrolidyl, 1-piperidinyl or 4-morpholinyl;
Y represents O or S;
Z represents O, S or N(R_{b}), R_{b} being hydrogen or alkyl;
or R₁ and R_{b} may be joined to form an imidazole or benzimidazole moiety; and the isomers and pharmaceutically acceptable salts of said compound.

30. A pharmaceutical composition for treating or preventing viral infections, said composition comprising an anti-viral agent, in an amount effective to attenuate viral infectivity, and a pharmaceutically acceptable carrier medium, said anti-viral agent comprising a compound of the formula: 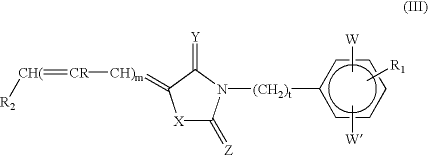
wherein
R represents hydrogen or alkyl; and m is an integer from 0-4;
R₁ represents hydrogen or a substituent selected from the group consisting of —OH, —COOR₄, —CONR₅R₆, —SO₂NR₇R₈, R₄, R₅, R₆, R₇ and R₈ being independently selected from the group of hydrogen or alkyl, or R₁ represents a mono-heterocylic radical selected from the group of furan, thiophene, pyridine, pyrimidine, pyridazine, 1,3-oxathiolane, tetrazole, oxadiazole, oxazole, isoxazole, triazole, imidazoline, imidazole, thiazole, thiadiazole, pyrrole, piperidine, morpholine, triazine and pyrazole;
W and W′ may be the same or different and represent hydrogen or a substituent selected from the group consisting of a straight or branched chain, saturated or unsaturated aliphatic group having 1-6 carbon atoms, halogen, nitro, hydroxy, perfluoroalkyl, difluoromethyl, alkoxy, phenoxy, phenylalkoxy, acyl, acyloxy, acyloxyalkyl, cyano, carbalkoxy, thio, alkylthio, alkylsulfinyl, alkylsulfonyl, amino, alkylamino, dialkylamino, sulfonamido, carboxamido and alkanoylamino;
t is an integer from 0 to 8;
X represents a moiety selected from the group consisting of —S—, —O— or —N(Rₐ)—, Rₐ being hydrogen or alkyl;
R₂ represents a radical selected from those consisting of an unsubstituted or substituted phenylalkyl radical, an unsubstituted or substituted phenylalkenyl radical, an unsubstituted or substituted phenylalkynyl radical, an unsubstituted or substituted biphenylalkyl radical, an unsubstituted or substituted polycyclic radical, an unsubstituted or substituted alycyclic radical having 5-8 carbon atoms or a radical of the formula (R₂ₐ—)ₙ(L—)ₚR_{2b}—, wherein R₂ₐ and R_{2b} may be the same or different and represent an unsubstituted or substituted heterocyclic radical or an unsubstituted or substituted phenyl radical, R₂ₐ also represents an unsubstituted or substituted polycyclic radical and L represents a divalent linking moiety selected from the group consisting of a valence bond, —(CH₂)_{q}—, —HC═CH—, —C═C—, —C(═O)—, —O—, —S—, —S(═O)—, —S(═O)₂—, NR_{2c}, R_{2c} being hydrogen or alkyl, n and p are each 0 or 1, and q is an integer from 1 to 3;
said heterocyclic radicals being selected from the group consisting of furan, thiophene, oxazole, oxadiazole, isoxazole, pyridine, pyrimidine, pyrazole, triazole, pyridazine, 1,3-oxathiolane, thiazole, isothiazole, thiadiazole, imidazole, pyrrole, tetrazole and triazine;
said polycyclic radicals being selected from the group consisting of benzofuran, isobenzofuran, benzothiophene, isobenzothiophene, benzoxazole, indole, 2-isoindole, benzopyrazole, quinoline, isoquinoline, 1,2-benzodiazine, 1,3-benzodiazine, 1,2,3-benzotriazole, benzothiazole, benzimidazole, 1,2,3-benzotriazine, 1,2,4-benzotriazine, naphthalene, anthracene and fluorene;
the heterocyclic radical substituents, said polycyclic radical substituents and said alicyclic radical substituents being at least one selected from the group consisting of a straight or branched chain, saturated or unsaturated aliphatic group having 1-6 carbon atoms, halogen, perhaloalkyl, monoalkyl, dihaloalkyl, alkoxy, acyl, acyloxy, acyloxyalkyl, phenylalkoxy, hydroxy, hydroxyalkyl, thio, alkylthio, nitro, carboxy, carbalkoxy;
the phenyl radical substituents, the phenylalkyl radical substituents, the phenylalkenyl radical substituents, the phenylalkynyl radical substituents and the biphenylalkyl radical substituents being at least one selected from the group consisting of a straight or branched chain, saturated or unsaturated aliphatic group having 1-6 carbon atoms, halogen, nitro, carboxy, hydroxy, hydroxyalkyl, perhaloalkyl, monohaloalkyl, dihaloalkyl, alkoxy, phenylalkoxy, acyl, acyloxy, acyloxyalkyl, cyano, carbalkoxy, thio, alkylthio, alkylsulfinyl, alkylsulfonyl, amino, alkylamino, dialkylamino, aminoalkyl, alkylaminoalkyl, dialkylaminoalkyl, sulfonamido, carboxamido, alkanoylamino.
Y represents O or S;
Z represents O, S or N(R_{b}), R_{b} being hydrogen or alkyl;
or R₁ and R_{b} may be joined to form an imidazole or benzimidazole moiety; and the isomers and pharmaceutically acceptable salts of said compound.

31. A composition as claimed in claim 30, wherein said R₂ radical is of the formula (R₂ₐ—)ₙ(L—)ₚR_{2b}—, p is 0; and m is 0.

32. A composition as claimed in claim 30 comprising a compound of formula III, wherein R₁ is a carboxyl group, W and W′ represent hydrogen, halogen, hydroxy, alkyl or trifluoromethyl substituents, Y is oxygen, X and Z are sulfur, R₂ represents an unsubstituted or substituted furan group or an unsubstituted or substituted thiophene group, the furan substituent(s) and the thiophene substituent(s) being at least one selected from those consisting of alkyl, monohalophenyl, dihalophenyl, monohalocarboxyphenyl, carboxyphenyl, trifluoromethylphenyl, monohalotrifluoromethylphenyl, phenylethynyl, monoalkylphenyl, dialkylphenyl, furanyl, and thienyl, m=0 and t=0.

33. A composition as claimed in claim 30 comprising a compound of formula III, wherein R₁ is a carboxyl group, W, and W′ represent hydrogen, halogen, hydroxy or trifluoromethyl substituents, Y is oxygen, X and Z are sulfur, R₂ represents an unsubstituted or substituted phenyl group, the phenyl substituent(s) being at least one selected from those consisting of halogen, alkoxy, carboxy, an unsubstituted or substituted 2-phenylethenyl group, an unsubstituted or substituted furan group, or an unsubstituted or substituted thiophene group, the 2-phenylethenyl substituent(s), the furan substituent(s) and the thiophene substituent(s) being at least one selected from those consisting of a straight or branched chain, saturated or unsaturated aliphatic group having 1-6 carbon atoms, halogen, nitro, carboxy, hydroxy, trifluoromethyl, difluoromethyl, alkoxy, phenoxy, phenylalkoxy, acyloxy, cyano, carbalkoxy, thio, alkylthio, alkylsulfinyl, alkylsulfonyl, amino, alkylamino, dialkylamino, sulfonamide, carboxamide or alkanoylamino, m=0 and t=0.

34. A composition as claimed in claim 30 comprising a compound of formula III selected from the group consisting of 4-(5-(5-[2-chloro-5-trifluoromethylphenyl]furan-2-yl-methylene)4-oxo-2-thionothiazolidin-3-yl)benzoic acid; 4-(5-(5-[3,4-dichlorophenyl]furan-2-yl-methylene)-4-oxo-2-thionothiazolidin-3-yl)benzoic acid; 4-(5-(5-[benzothiophen-2-yl]furan-2-yl-methylene)-4-oxo-2-thionothiazolidin-3-yl)benzoic acid; 4-(5-(5-[benzofuran-2-yl]furan-2-yl-methylene)-4-oxo-2-thionothiazolidin-3-yl)benzoic acid; 4-(5-{5-[3-bromo-6-methoxyphenyl]furan-2-yl-methylene}-4-oxo-2-thionothiazolidin-3-yl)benzoic acid; 4-(5-(5-[5-chlorothiophen-2-yl]thiazol-2-yl-methylene)-4-oxo-2-thionothiazolidin-3-yl)benzoic acid; 4-(5-(5-[3,5-ditrifluoromethylphenyl]furan-2-yl-methylene)-4-oxo-2-thionothiazolidin-3-yl)benzoic acid.

35. A composition as claimed in claim 30, further including at least one therapeutic agent selected from the group consisting of interferons, ribavirin, protease inhibitors, immunoglobulins, immunomodulators, hepatoprotectants, anti-inflammatory agents, antibiotics, antivirals and anti-infectious agents.

36. A pharmaceutical composition for treating or preventing viral infections, said composition comprising an anti-viral agent in an amount effective to attenuate viral infectivity, and a pharmaceutically acceptable carrier medium, said anti-viral agent comprising a compound of the formula: 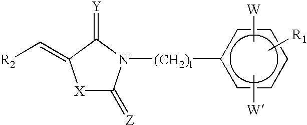
wherein
R₁ represents hydrogen or a substituent selected from the group consisting of —OH, —COOR₃, —CONR₄R₅, —SO₂NR₆R₇, R₃, R₄, R₅, R₆ and R₇ being independently selected from the group of hydrogen, alkyl, or R₁ represents a heterocylic ring selected from the group of tetrazole, oxadiazole, oxazole, triazole, imidazoline, imidazole, thiazole, thiadiazole, pyrrole, piperidine, morpholine and pyrazole;
W and W′ may be the same or different and represent hydrogen or a substituent selected from the group consisting of a straight or branched chain, saturated or unsaturated aliphatic group having 1-6 carbon atoms, halogen, nitro, hydroxy, perfluoroalkyl, difluoromethyl, alkoxy, phenoxy, acyloxy, cyano, carbalkoxy, thio, alkylthio, alkylsulfinyl, alkylsulfonyl, amino, alkylamino, dialkylamino, sulfonamide, carboxamide and alkanoylamino.
t is an integer from 0 to 8;
X represents a moiety selected from the group consisting of —S—, —O— or —N(Rₐ)—, Rₐ being hydrogen or alkyl;
R₂ represents a radical selected from those consisting of an unsubstituted or substituted mono- or bi-heterocyclic group, an unsubstituted or substituted polycyclic ring moiety, an unsubstituted or substituted alicyclic group having 5-8 carbon atoms, an unsubstituted or substituted phenyl group, an unsubstituted or substituted biphenyl (C₆H₅—C₆H₄—) group, an unsubstituted or substituted phenyl ether group (C₆H₅—O—C₆H₄—), an unsubstituted or substituted cinnamyl (C₆H₅CH═CH—) group, or an unsubstituted or substituted cinnamylphenyl group, said mono-heterocyclic group being selected from those consisting of furan, thiophene, oxazole, oxadiazole, pyridine, pyrimidine, pyrazole, triazole, pyridazine, 1,3-oxathiolane, thiazole, thiadiazole, imidazole, tetrazole, pyrrole and triazine, said bi-heterocyclic group comprising two heterocyclic groups, said two heterocyclic groups being selected from said mono-heterocyclic groups and being the same or different, said polycyclic ring moiety being selected from those consisting of benzofuran, isobenzofuran, benzothiophene, isobenzothiophene, benzoxazole, benzopyrrole, indolenine, 2-isobenzazole, benzpyrazole, quinoline, isoquinoline, 1,2-benzodiazine, 1,3-benzodiazine, 1,2,3-benzotriazole, benzothiazole, benzimidazole, 1,2,3-benzotriazine, 1,2,4-benzotriazine, naphthalene, anthracene and fluorene;
the mono-heterocyclic group substituents, the bi-heterocyclic group substituents, the alicyclic group substituents and the polycyclic ring moiety substituents being at least one selected from those consisting of a straight or branched chain, saturated or unsaturated aliphatic group having 1-6 carbon atoms, halogen, trifluoromethyl, alkoxy, hydroxy, thio, nitro, carboxy, carbalkoxy, an unsubstituted or substituted phenyl group, an unsubstituted or substituted phenylalkyl group, an unsubstituted or substituted phenylalkenyl group or an unsubstituted or substituted phenylalkynyl group;
the phenyl group substituents, the biphenyl group substituents, the phenyl ether group substituents, the phenylalkyl group substituents, the phenylalkenyl group substituents, the phenylalkynyl group substituents, the cinnamyl group substituents and the cinnamylphenyl group substituents being at least one selected from those consisting of a straight or branched chain, saturated or unsaturated aliphatic group having 1-6 carbon atoms, halogen, nitro, carboxy, hydroxy, trifluoromethyl, difluoromethyl, alkoxy, phenoxy, acyloxy, cyano, carbalkoxy, thio, alkylthio, alkylsulfinyl, alkylsulfonyl, amino, alkylamino, dialkylamino, sulfonamido, carboxamido, alkanoylamino, 1-pyrrolidyl, 1-piperidinyl or 4-morpholinyl;
Y represents O or S;
Z represents O, S or N(R_{b}), R_{b} being hydrogen or alkyl;
or R₁ and R_{b} may be joined to form an imidazole or benzimidazole moiety; and the isomers and pharmaceutically acceptable salts of said compound.

37. A compound having the formula: 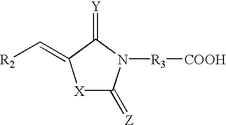
wherein
R₃ represents an unsubstituted or substituted, branched or straight chain, saturated or unsaturated hydrocarbon moiety having 1-10 carbon atoms in the main chain, said hydrocarbon moiety substituents being at least one selected from those consisting of branched or straight chain, saturated or unsaturated aliphatic group, having 1-6 carbon atoms, unsubstituted or substituted mono-heterocyclic group or unsubstituted or substituted phenyl (C₆H₅) group, said mono-heterocyclic group being selected from those consisting of furan, thiophene, oxazole, oxadiazole, pyridine, pyrimidine, pyrazole, triazole, pyridazine, 1,3-oxathiolane, thiazole, thiadiazole, imidazole, tetrazole, pyrrole and triazine;
X represents a moiety selected from the group consisting of —S—, —O— or —N(Rₐ)—, Rₐ being hydrogen or alkyl;
R₂ represents a radical selected from those consisting of an unsubstituted or substituted mono- or bi-heterocyclic radical, an unsubstituted or substituted polycyclic radical, an unsubstituted or substituted polycyclic-heterocyclic radical, an unsubstituted or substituted alicyclic radical having 5-8 carbon atoms, an unsubstituted or substituted phenyl radical, an unsubstituted or substituted biphenyl (C₆H₅—C₆H₄—) radical, an unsubstituted or substituted phenyl ether (C₆H₅—O—C₆H₄—) radical or an unsubstituted or substituted 2-phenylethenyl (C₆H₅CH═CH—) radical, said mono-heterocyclic group being selected from those consisting of furan, thiophene, oxazole, oxadiazole, pyridine, pyrimidine, pyrazole, triazole, pyridazine, 1,3-oxathiolane, thiazole, thiadiazole, imidazole, tetrazole, pyrrole and triazine, said bi-heterocyclic group comprising two heterocyclic moieties which are selected from said mono-heterocyclic radical group members, and which may be the same or different, said polycyclic ring moiety being selected from those consisting of benzofuran, isobenzofuran, benzothiophene, isobenzothiophene, benzoxazole, indole, 2-isoindole, benzopyrazole, quinoline, isoquinoline, 1,2-benzodiazine, 1,3-benzodiazine, 1,2,3-benzotriazole, benzothiazole, benzimidazole, 1,2,3-benzotriazine and 1,2,4-benzotriazine, naphthalene, anthracene and fluorene and said polycyclic-heterocyclic radical comprising a polycyclic moiety selected from said polycyclic radical group members and a heterocyclic moiety which is selected from said mono-heterocyclic radical group members;
the mono-heterocyclic radical substituents, the bi-heterocyclic radical substituents, the alicyclic radical substituents, the polycyclic radical substituents and the polycyclic-heterocyclic radical substituents being at least one selected from those consisting of a straight or branched chain, saturated or unsaturated aliphatic group having 1-6 carbon atoms, halogen, trifluoromethyl, alkoxy, hydroxy, thio, nitro, carbalkoxy, an unsubstituted or substituted phenyl radical, an unsubstituted or substituted phenylalkenyl radical or an unsubstituted or substituted phenylalkynyl radical;
the phenyl radical substituents, the biphenyl radical substituents, the phenyl ether radical substituents, the phenylalkenyl radical substituents, the phenylalkynyl radical substituents and the 2-phenylethenyl radical substituents being at least one selected from those consisting of a straight or branched chain, saturated or unsaturated aliphatic group having 1-6 carbon atoms, halogen, nitro, carboxy, hydroxy, trifluoromethyl, difluoromethyl, alkoxy, phenoxy, acyloxy, cyano, carbalkoxy, thio, alkylthio, alkylsulfinyl, alkylsulfonyl, amino, alkylamino, dialkylamino, sulfonamido, carboxamido, alkanoylamino, 1-pyrrolidyl, 1-piperidinyl or 4-morpholinyl;
Y represents O or S;
Z represents O, S or N(R_{b}), R_{b} being hydrogen or alkyl;
or R₁ and R_{b} may be joined to form an imidazole or benzimidazole moiety; and the isomers and pharmaceutically acceptable salts of said compound.

38. A compound as claimed in claim 37, wherein R₁ is a straight chain alkylene of 1-5 carbon atoms, Y is oxygen, X and Z are sulfur and R₂ is an unsubstituted or substituted mono-heterocyclic group selected from those consisting of furan, thiophene and oxazole, or an unsubstituted or substituted bi-heterocyclic group selected from those consisting of bi-thienyl and 1H-pyrazolylthienyl, the heterocyclic group substituents being at least one halogen, trifluoromethyl or an unsubstituted or substituted phenyl group, said phenyl group substituents being at least one selected from those consisting of halogen, nitro, carboxy, hydroxy,methyl, ethyl, trifluoromethyl, difluoromethyl, alkoxy, phenoxy, acyloxy, cyano, carbalkoxy, thioalkylthio, alkylsulfinyl, alkylsulfonyl, amino, alkylamino, dialkylamino, sulfonamido, carboxamido, alkanoylamino, 1-pyrrolidyl, 1-piperidinyl or 4-morpholinyl.

39. A compound as claimed in claim 37, wherein R₁ is a straight chain alkylene of 1-5 carbon atoms, Y is oxygen, X and Z are sulfur and R₂ is an unsubstituted or substituted phenyl group the phenyl substituents being at least one selected from those consisting of halogen, nitro, carboxy, hydroxy,methyl, ethyl, trifluoromethyl, difluoromethyl, alkoxy, phenoxy, acyloxy, cyano, carbalkoxy, thioalkylthio, alkylsulfinyl, alkylsulfonyl, amino, alkylamino, dialkylamino, sulfonamido, carboxamido, alkanoylamino, 1-pyrrolidyl, 1-piperidinyl or 4-morpholinyl.

40. A compound as claimed in claim 37, wherein R₁ is a straight chain alkylene of 1-5 carbon atoms, Y is oxygen, X and Z are sulfur and R₂ is an unsubstituted or substituted polycyclic ring moiety selected from those consisting of 9-phenanthryl and 2-fluorenyl, said polycyclic ring moiety substituents being at least one selected from those consisting of methyl, ethyl, halogen, alkoxy, hydroxy, thio, nitro or an unsubstituted or substituted phenyl group, the phenyl group substituents being at least one selected from those consisting of halogen, nitro, carboxy, hydroxy,methyl, ethyl, trifluoromethyl, difluoromethyl, alkoxy, phenoxy, acyloxy, cyano, carbalkoxy, thioalkylthio, alkylsulfinyl, alkylsulfonyl, amino, alkylamino, dialkylamino, sulfonamido, carboxamido, alkanoylamino, 1-pyrolidyl, piperidinyl or 4-morpholinyl.

41. A compound as claimed in claim 37, selected from the group of
(5-[(5-{3,4-dichlorophenyl}furan-2-yl)methylene]-4-oxo-2-thionothiazolidin-3-yl)acetic acid;
(5-[(5-{2-chloro-5-trifluoromethylphenyl}furan-2-yl)methylene]-4-oxo-2-thionothiazolidin-3-yl)acetic acid;
(5-[(5-{3-trifluoromethylphenyl}furan-2-yl)methylene]-4-oxo-2-thionothiazolidin-3-yl)acetic acid;
(5-[(5-{3,5-dichlorophenyl}furan-2-yl)methylene]-4-oxo-2-thionothiazolidin-3-yl)acetic acid;
(5-[(5-{4-chlorophenyl}furan-2-yl)methylene]-4-oxo-2-thionothiazolidin-3-yl)acetic acid;
(5-[(5-{4-chlorophenyl-3-ethoxycarbonyl}furan-2-yl)methylene]-4-oxo-2-thionothiazolidin-3-yl)acetic acid;
(5-[(5-{3,4-dichlorophenyl}thiophen-2-yl)methylene]-4-oxo-2-thionothiazolidin-3-yl)acetic acid;
(5-[(5-{3-t-butylphenoxyphenyl}furan-2-yl)methylene]-4-oxo-2-thionothiazolidin-3-yl)acetic acid.

42. A compound as claimed in claim 37, selected from the group of
2-(5-[(5-{2-chloro-5-trifluoromethylphenyl}furan-2-yl)methylene]-4-oxo-2-thionothiazolidin-3-yl)propionic acid;
2-(5-[(5-{5-chlorothiophen-2-yl}furan-2-yl)methylene]-4-oxo-2-thionothiazolidin-3-yl)propionic acid;
3-(5-[(5-{benzofuran-2-yl}furan-2-yl)methylene]-4-oxo-2-thionothiazolidin-3-yl)propionic acid;
3-(5-[(5-{benzothiophen-2-yl}furan-2-yl)methylene]-4-oxo-2-thionothiazolidin-3-yl)propionic acid;
3-(5-[(5-{2-chloro-5-trifluoromethylphenyl}furan-2-yl)methylene]-4-oxo-2-thionothiazolidim-3-yl)propionic acid;
3-(5-[(5-{3,5-ditrifluoromethylphenyl}furan-2-yl)methylene]-4-oxo-2-thionothiazolidin-3-yl)propionic acid;
3-(5-[(5-{furan-2-yl}furan-2-yl)methylene]-4-oxo-2-thionothiazolidin-3-yl)propionic acid;
3-(5-[(5-{thiophen-2-yl}furan-2-yl)methylene]-4-oxo-2-thioxothiazolidin-3-yl)propionic acid;
3-(5-[(5-{5-chlorothiophen-2-yl}furan-2-yl)methylene]-4-oxo-2-thionothiazolidin-3-yl)propionic acid;
3-(5-[(-{4-bromophenyl}furan-2-yl)methylene]-4-oxo-2-thionothiazolidin-3-yl)propionic acid;
3-(5-[(5-{isoquinolin-2-yl}furan-2-yl)methylene]-4-oxo-2-thionothiazolidin-3-yl)propionic acid;
3-(5-[(5-{2-trifluoromethylphenyl}furan-2-yl)methylene]-4-oxo-2-thionothiazolidin-3-yl)propionic acid;
3-(5-[(5-{3,4-methylenedioxyphenyl}furan-2-yl)methylene]-4-oxo-2-thionothiazolidin-3-yl)propionic acid;
3-(5-[(5-{3,5-dichlorophenyl}furan-2-yl)methylene]-4-oxo-2-thionothiazolidin-3-yl)propionic acid;
3-(5-[(5-{3,4-dichlorophenyl}furan-2-yl)methylene]-4-oxo-2-thionothiazolidin-3-yl)propionic acid;
3-(5-[(5-{3,5-dimethylphenyl}furan-2-yl)methylene]-4-oxo-2-thionothiazolidin-3-yl)propionic acid;
3-(5-[(5-{5-methylthiophen-2-yl}furan-2-yl)methylene]-4-oxo-thionothiazolidin-3-yl)propionic acid;
3-(5-[(5-{5-methyl-2-pyridyl}furan-2-yl)methylene]-4-oxo-2-thionothiazolidin-3-yl)propionic acid;
3-(5-[(6-benzyloxybenzofuran-2-yl)methylene]-4-oxo-2-thionothiazolidin-3-yl)propionic acid;
3-(5-[(5-{phenanthren-9-yl}furan-2-yl)methylene]-4-oxo-2-thionothiazolidin-3-yl)propionic acid;
3-(5-[(5-{thiophen-2-yl}thiophen-2-yl)methylene]-4-oxo-2-thionothiazolidin-3-yl)propionic acid;
3-(5-[(5-{fluorene-2-yl}furan-2-yl)methylene]-4-oxo-2-thionothiazolidin-3-yl)propionic acid;
3-(5-[(5-{phenylethynyl}thiophen-2-yl)methylene]-4-oxo-2-thionothiazolidin-3-yl)propionic acid;
3-(5-[(5-{3-chlorophenyl}furan-2-yl)methylene]-4-oxo-2-thionothiazolidin-3-yl)propionic acid;
3-(5-[(4-{phenylethynyl}thiophen-2-yl)methylene]-4-oxo-2-thionothiazolidin-3-yl)propionic acid;
3-(5-[(5-{5-n-propylthiophen-2-yl}furan-2-yl)methylene]-4-oxo-2-thionothiazolidin-3-yl)propionic acid; and
3-(5-[(5-{4-chlorophenyl}furan-2-yl)methylene]-4-oxo-2-thionothiazolidin-3-yl)propionic acid.

43. The compound 3-(5-[(5-{benzofuran-2-yl}furan-2-yl)methylene]-4-oxo-2-thionothiazolidin-3-yl)propionic acid, according to claim 37.

44. The compound 3-(5-[(5-{benzothiophen-2-yl}furan-2-yl)methylene]-4-oxo-2-thionothiazolidin-3-yl)propionic acid, according to claim 37.

45. The compound 3-(5-[(5-{2-chloro-5-trifluoromethylphenyl}furan-2-yl)methylene]-4-oxo-2-thionothiazolidin-3-yl)propionic acid, according to claim 37.

46. The compound 3-(5-[(5-{5-chlorothiophen-2-yl}furan-2-yl)methylene]-4-oxo-2-thionothiazolidin-3-yl)propionic acid, according to claim 37.

47. A compound having the formula: 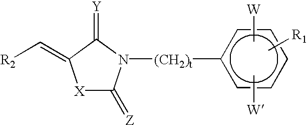
wherein
R₁ represents hydrogen or a substituent selected from the group consisting of —OH, —COOH, —CONR₄R₅, —SO₂NR₆R₇, R₄, R₅, R₆ and R₇ being independently selected from the group of hydrogen, alkyl, or R₁ represents a heterocylic ring selected from the group of furan, thiophene, pyridine, pyrimidine, pyridazine, 1,3-oxathiolane, tetrazole, oxadiazole, oxazole, triazole, imidazoline, imidazole, thiazole, thiadiazole, pyrrole, piperidine, morpholine, triazine and pyrazole;
W and W′ may be the same or different and represent hydrogen or a substituent selected from the group consisting of a straight or branched chain, saturated or unsaturated aliphatic group having 1-6 carbon atoms, halogen, nitro, hydroxy, perfluoroalkyl, difluoromethyl, alkoxy, phenoxy, acyloxy, cyano, carbalkoxy, thio, alkylthio, alkylsulfinyl, alkylsulfonyl, amino, alkylamino, dialkylamino, sulfonamido, carboxamido and alkanoylamino.
t is an integer from 0 to 8;
X represents a moiety selected from the group consisting of —S—, —O— or —N(Rₐ)—, Rₐ being hydrogen or alkyl;
R₂ represents a radical selected from those consisting of an unsubstituted or substituted mono- or bi-heterocyclic radical, an unsubstituted or substituted polycyclic radical, an unsubstituted or substititued polycyclic-heterocyclic radical, an unsubstituted or substituted alicyclic radical having 5-8 carbon atoms, an unsubstituted or substituted phenyl radical, an unsubstituted or substituted biphenyl (C₆H₅—C₆H₄—) radical, an unsubstituted or substituted phenyl ether (C₆H₅—O—C₆H₄—) radical, an unsubstituted or substituted 2-phenylethenyl (C₆H₅CH═CH—) radical, or an unsubstituted or substituted stilbenyl (C₆H₅—CH═CH—C₆H₄—) radical, said mono-heterocyclic radical being selected from those consisting of furan, thiophene, oxazole, oxadiazole, pyridine, pyrimidine, pyrazole, triazole, pyridazine, 1,3-oxathiolane, thiazole, thiadiazole, imidazole, tetrazole, pyrrole and triazine, said bi-heterocyclic group comprising two heterocyclic groups, said two heterocyclic groups being selected from said mono-heterocyclic radical group members and being the same or different, said polycyclic radical being selected from the group consisting of benzofuran, isobenzofuran, benzothiophene, isobenzothiophene, benzoxazole, benzopyrrole, 2-isoindole, benzopyrazole, quinoline, isoquinoline, 1,2-benzodiazine, 1,3-benzodiazine, 1,2,3-benzotriazole, benzothiazole, benzimidazole, 1,2,3-benzotriazine, 1,2,4-benzotriazine, naphthalene, anthracene and fluorene, and said polycyclic-heterocyclic radical comprising a polycyclic moiety selected from said polycyclic radical group members and a heterocyclic moiety selected from said mono-heterocyclic radical group members;
the mono-heterocyclic radical substituents, the bi-heterocyclic radical substituents, the alicyclic radical substituents, the polycyclic radical substituents and the polycyclic-heterocyclic radical substituents being at least one selected from those consisting of a straight or branched chain, saturated or unsaturated aliphatic group having 1-6 carbon atoms, halogen, trifluoromethyl, alkoxy, hydroxy, thio, nitro, acyl, carboxy, carbalkoxy, an unsubstituted or substituted phenyl radical, an unsubstituted or substituted phenylalkyl radical, an unsubstituted or substituted phenylalkenyl radical or an unsubstituted or substituted phenylalkynyl radical;
the phenyl radical substituents, the biphenyl radical substituents, the phenyl ether radical substituents, the phenylalkyl radical substituents, the phenylalkenyl radical substituents, the phenylalkynyl radical substituents, the 2-phenylethenyl radical substituents and the stilbenyl radical substituents being at least one selected from those consisting of a straight or branched chain, saturated or unsaturated aliphatic group having 1-6 carbon atoms, halogen, nitro, carboxy, hydroxy, trifluoromethyl, difluoromethyl, alkoxy, phenoxy, phenylalkoxy, acyl, acyloxy, cyano, carbalkoxy, thio, alkylthio, alkylsulfinyl, alkylsulfonyl, amino, alkylamino, dialkylamino, sulfonamido, carboxamido, alkanoylamino, furan, thiophene, pyridine, pyrimidine, pyridazine, 1,3-oxathiolane, tetrazole, oxadiazole, oxazole, triazole, imidazoline, imidazole, thiazole, thiadiazole, pyrrole, piperidine, morpholine and pyrazole;
Y represents O or S;
Z represents O, S or N(R_{b}), R_{b} being hydrogen or alkyl;
or R₁ and R_{b} may be joined to form an imidazole or benzimidazole moiety; and the isomers and pharmaceutically acceptable salts of said compound.

48. A compound as claimed in claim 47, wherein R₁ is a carboxyl group, W and W′ represent hydrogen, halogen, hydroxy, alkyl or trifluoromethyl substituents, Y is oxygen, X and Z are sulfur, R₂ represents an unsubstituted or substituted furan group or an unsubstituted or substituted thiophene group, the furan substituent(s) and the thiophene substituent(s) being at least one selected from those consisting of alkyl, monohalophenyl, dihalophenyl, monohalocarboxyphenyl, carboxyphenyl, trifluoromethylphenyl, monohalotrifluoromethylphenyl, phenylethynyl, monoalkylphenyl, dialkylphenyl, furanyl and thienyl and t=0.

49. A compound as claimed in claim 47, wherein R₁ is a carboxyl group, W, and W′ represent hydrogen, halogen, hydroxy or trifluoromethyl substituents, Y is oxygen, X and Z are sulfur, R₂ represents an unsubstituted or substituted phenyl group, the phenyl substituent(s) being at least one selected from those consisting of halogen, alkoxy, carboxy, an unsubstituted or substituted 2-phenylethenyl group, an unsubstituted or substituted furan group, or an unsubstituted or substituted thiophene group, the 2-phenylethenyl substituent(s), the furan substituent(s) and the thiophene substituent(s) being at least one selected from those consisting of a straight or branched chain, saturated or unsaturated aliphatic group having 1-6 carbon atoms, halogen, nitro, carboxy, hydroxy, trifluoromethyl, difluoromethyl, alkoxy, phenoxy, phenylalkoxy, acyloxy, cyano, carbalkoxy, thio, alkylthio, alkylsulfinyl, alkylsulfonyl, amino, alkylamino, dialkylamino, sulfonamide, carboxamide or alkanoylamino and t=0.

50. A compound as claimed in claim 47, selected from the group consisting of
4-(5-[5-{benzofuran-2-yl}furan-2-yl)methylene]-4-oxo-2-thionothiazolidin-3-yl)benzoic acid;
4-(5-[(5-{benzothiophen-2-yl}furan-2-yl)methylene]-4-oxo-2-thionothiazolidin-3-yl)benzoic acid;
4-(5-[(5-{3,5-ditrifluoromethylphenyl}furan-2-yl)methylene]-4-oxo-2-thionothiazolidin-3-yl)benzoic acid;
4-(5-[(5-{2-chloro-5-trifluoromethylphenyl}furan-2-yl)methylene]-4-oxo-2-thionothiazolidin-3-yl)benzoic acid;
4-(5-[(5-{3,4-dimethylphenyl}furan-2-yl)methylene]-4-oxo-2-thionothiazolidin-3-yl)benzoic acid;
4-(5-[(5-{5-chlorothiophen-2-yl}furan-2-yl)methylene]-4-oxo-2-thionothiazolidin-3-yl)benzoic acid;
4-(5-[(5-{2,5-dichlorophenyl}furan-2-yl)methylene]-4-oxo-2-thionothiazolidin-3-yl)benzoic acid;
4-(5-[(5-{2-chlorophenyl}furan-2-yl)methylene]-4-oxo-2-thionothiazolidin-3-yl)benzoic acid;
4-(5-[(5-{2-furanyl}furan-2-yl)methylene]-4-oxo-2-thionothiazolidin-3-yl)benzoic acid;
4-(5-[(5-{5-methylpyridin-2-yl}furan-2-yl)methylene]-4-oxo-2-thionothiazolidin-3-yl)benzoic acid;
4-(5-[(5-{5-methylthiophen-2-yl}furan-2-yl)methylene]-4-oxo-2-thionothiazolidin-3-yl)benzoic acid;
4-(5-[(5-{3,4-difluorophenyl}furan-2-yl)methylene]-4-oxo-2-thionothiazolidin-3-yl)benzoic acid;
4-(5-[(5-{4-methoxyphenyl}furan-2-yl)methylene]-4-oxo-2-thionothiazolidin-3-yl)benzoic acid;
4-(5-[(5-{5-acetothiophen-2-yl}furan-2-yl)methylene]-4-oxo-2-thionothiazolidin-3-yl)benzoic acid;
4-(5-[(5-{3-chloro-5-trifluoromethylpyridin-2-yl}furan-2-yl)methylene]-4-oxo-2-thionothiazolidin-3-yl)benzoic acid;
4-(5-[(5-{3,4-dimethoxyphenyl}furan-2-yl)methylene]-4-oxo-2-thionothiazolidin-3-yl)benzoic acid;
4-(5-[(5-{3,4-methylenedioxyphenyl}furan-2-yl)methylene]-4-oxo-2-thionothiazolidin-3-yl)benzoic acid;
4-(5-[(5-{5-trifluoromethylpyridin-2-yl}furan-2-yl)methylene]-4-oxo-2-thionothiazolidin-3-yl)benzoic acid;
4-(5-[(5-{6-methoxypyridazin-3-yl}furan-2-yl)methylene]-4-oxo-2-thionothiazolidin-3-yl)benzoic acid;
4-(5-[(5-{4,6-dimethylpyridin-2-yl}furan-2-yl)methylene]-4-oxo-2-thionothiazolidin-3-yl)benzoic acid;
4-(5-[(5-{3-bromo-6-methoxyphenyl}furan-2-yl)methylene]-4-oxo-2-thionothiazolidin-3-yl)benzoic acid;
4-(5-[(5-phenylethynylfuran-2-yl)methylene]-4-oxo-2-thionothiazolidin-yl)benzoic acid
4-(5-[(5-{3,4-dichlorophenyl}furan-2-yl)methylene]-4-oxo-2-thionothiazolidin-3-yl)benzoic acid;
4-(5-[(5-{4-chlorophenyl}furan-2-yl)methylene]-4-oxo-2-thionothiazolidin-3-yl)benzoic acid;
4-(5-[(5-{4-bromophenyl}furan-2-yl)methylene]-4-oxo-2-thionothiazolidin-3-yl)benzoic acid;
4-(5-[(5-{3,5-dichlorophenyl}furan-2-yl)methylene]-4-oxo-2-thionothiazolidin-3-yl)benzoic acid;
4-(5-[(5-{2-chloro-5-trifluoromethylphenyl}furan-2-yl)methylene]-4-oxo-2-thionothiazolidin-3-yl)-6-chlorobenzoic acid;
4-(5-[(5-{3-carboxyphenyl}furan-2-yl)methylene]-4-oxo-2-thionothiazolidin-3-yl)benzoic acid;
4-(5-[(5-{2-trifluoromethylphenyl}furan-2-yl)methylene]-4-oxo-2-thionothiazolidin-3-yl)benzoic acid;
4-(5-[(5-{2,3,5,6-tetrafluoropyridin-4-yl}furan-2-yl)methylene]-4-oxo-2-thionothiazolidin-3-yl)benzoic acid;
4-(5-[(5-{3-trifluoromethylphenyl}furan-2-yl)methylene]-4-oxo-2-thionothiazolidin-3-yl)benzoic acid;
4-(5-[(5-{2-thienyl}furan-2-yl)methylene]-4-oxo-2-thionothiazolidin-3-yl)benzoic acid;
4-(5-[(5-{4-n-butylphenyl}furan-2-yl)methylene]-4-oxo-2-thionothiazolidin-3-yl)benzoic acid;
4-(5-[(5-{2-chloro-5-trifluoromethylphenyl}furan-2-yl)methylene]-4-oxo-2-thionothiazolidin-3-yl)methylenebenzoic acid;
4-(5-[(5-{3,5-difluorophenyl}furan-2-yl)methylene]-4-oxo-2-thionothiazolidin-3-yl)benzoic acid;
4-(5-[(5-{3,5-dimethylphenyl}furan-2-yl)methylene]-4-oxo-2-thionothiazolidin-3-yl)benzoic acid;
4-(5-[(5-{4-acetophenyl}furan-2-yl)methylene]-4-oxo-2-thionothiazolidin-3-yl)benzoic acid;
4-(5-[(5-{4-n-propylphenyl}furan-2-yl)methylene]-4-oxo-2-thionothiazolidin-3-yl)benzoic acid;
4-(5-[(5-{2,3-dichlorophenyl}furan-2-yl)methylene]-4-oxo-2-thionothiazolidin-3-yl)benzoic acid;
4-(5-[(5-{indol-2-yl}furan-2-yl)methylene]-4-oxo-2-thionothiazolidin-3-yl)benzoic acid;
4-(5-[(5-{3-methoxy-2-(N,N-diethylaminocarbonylphenyl)furan-2-yl)methylene]-4-oxo-2-thionothiazolidin-3-yl)benzoic acid;
4-(5-[(5-{phenyl}furan-2-yl)methylene]-4-oxo-2-thionothiazolidin-3-yl)benzoic acid;
4-(5-[(5-{5-methylpyridin-2-yl}furan-2-yl)methylene]-4-oxo-2-thionothiazolidin-3-yl)benzoic acid;
4-(5-[(5-{5-chloro-3-methylbenzothiophen-2-yl}furan-2-yl)methylene]-4-oxo-2-thionothiazolidin-3-yl)benzoic acid;
4-(5-[(5-{5-n-propylthiophen-2-yl}furan-2-yl)methylene]-4-oxo-2-thionothiazolidin-3-yl)benzoic acid;
4-(5-[(5-{4,5-dimethylfuran-2-yl}furan-2-yl)methylene]-4-oxo-2-thionothiazolidin-3-yl)benzoic acid;
4-(5-[(5-{5-thiazol-2-yl}furan-2-yl)methylene]-4-oxo-2-thionothiazolidin-3yl)benzoic acid;
4-(5-[(5-{formyl}furan-2-yl)methylene]-4-oxo-2-thionothiazolidin-3-yl)benzoic acid;
4-(5-[(5-{4-methylpyridin-2-yl}furan-2-yl)methylene]-4-oxo-2-thionothiazolidin-3-yl)benzoic acid;
4-(5-[(5-{2-acetophenyl}furan-2-yl)methylene]-4-oxo-2-thionothiazolidin-3-yl)benzoic acid;
4-(5-[(5-(2-nitrophenyl}furan-2-yl)methylene]-4-oxo-2-thionothiazolidin-3-yl)benzoic acid;
4-(5-[(5-{5-chlorothiophen-2-yl}thiazol-2-yl)methylene]-4-oxo-2-thionothiazolidin-3-yl)benzoic acid; and
4-(5-[(5-{4,5-dichloroimidazol-2-yl}furan-2-yl)methylene]-4-oxo-2-thionothiazolidin-3-yl)benzoic acid.

51. A compound as claimed in claim 47, selected from the group consisting of
3-(5-[(5-{2-chloro-5-trifluoromethylphenyl}furanyl-2-yl)methylene]-4-oxo-2-thionothiazolidin-3-yl)-4-chlorobenzoic acid;
3-(5-[(5-{2-chloro-5-trifluoromethylphenyl}furanyl-2-yl)methylene]-4-oxo-2-thionothiazolidin-3-yl)-benzoic acid;
2-(5-[(5-{2-chloro-5-trifluoromethylphenyl}furanyl-2-yl)methylene]-4-oxo-2-thionothiazolidin-3-yl)-benzoic acid;
3-(5-[(5-{2-methyl-5-trifluoromethylpyrazol-3-yl}thiophen-2-yl)methylene]-4-oxo-2-thionothiazolidin-3-yl)benzoic acid;
3-(5-[(5-{2-trifluoromethylphenyl}furanyl-2-yl)methylene]-4-oxo-2-thionothiazolidin-3-yl)benzoic acid.

52. The compound 4-(5-[(5-{2-chloro-5-trifluoromethylphenyl}furan-2-yl)methylene]-4-oxo-2-thionothiazolidin-3-yl)benzoic acid, according to claim 47.

53. The compound 4-(5-[(5-{3,4-dichlorophenyl}furan-2-yl)methylene]-4-oxo-2-thionothiazolidin-3-yl)benzoic acid, according to claim 47.

54. The compound 4-(5-[(5-{benzothiophen-2-yl}furan-2-yl)methylene]-4-oxo-2-thionothiazolidin-3-yl)benzoic acid, according to claim 47.

55. The compound 4-(5-[(5-{benzofuran-2-yl}furan-2-yl)methylene]-4-oxo-2-thionothiazolidin-3-yl)benzoic acid, according to claim 47.

56. The compound 4-(5-[(5-{3-bromo-6-methoxyphenyl}furan-2-yl)methylene]-4-oxo-2-thionothiazolidin-3-yl)benzoic acid, according to claim 47.

57. The compound 4-(5-[(5-{5-chlorothiophen-2-yl}thiazol-2-yl)methylene]-4-oxo-2-thionothiazolidin-3-yl)benzoic acid, according to claim 47.

58. The compound 4-(5-[(5-{3,5-ditrifluoromethylphenyl}furan-2-yl)methylene]-4-oxo-2-thionothiazolidin-3-yl)benzoic acid, according to claim 47.

59. The compound 4-(5-[(5-{2-chloro-5-trifluoromethylphenyl}furanyl-2-yl)methylene]-4-oxo-2-thionothiazolidin-3-yl)-2,6-difluorophenol, according to claim 47.
